(19) United States
(12) Patent Application Publication
(10) Pub. No.: US 2018/0028138 A1
(43) Pub. Date: Feb. 1, 2018

(54) MEDICAL IMAGE PROCESSING APPARATUS AND MEDICAL IMAGE PROCESSING METHOD

(71) Applicant: Samsung Electronics Co., Ltd., Gyeonggi-do (KR)

(72) Inventors: Do-kwan Oh, Suwon-si (KR); Byeong-won Lee, Suwon-si (KR); Choong-hwan Choi, Seongnam-si (KR)

(21) Appl. No.: 15/552,217

(22) PCT Filed: Feb. 24, 2016
(86) PCT No.: PCT/KR2016/001804

§ 371 Date: Aug. 18, 2017

(30) Foreign Application Priority Data
Feb. 24, 2015 (KR) 10-2015-0025909
Feb. 17, 2016 (KR) 10-2016-0018544

(51) Int. Cl. A61B 6/00; A61B 6/04

(52) U.S. Cl. CPC A61B 6/5235; A61B 6/461; A61B 6/469; A61B 6/5205; A61B 6/4464; A61B 6/4405; A61B 6/04; A61B 6/582

(57) ABSTRACT

A medical image processing apparatus includes a processor configured to acquire a first image and a second image captured by radiating an X ray to an object, generate a synthesis image by overlapping a first region of the first image with a second region of the second image, and determine matching accuracy representing a degree to which the first region and the second region match with each other; and a display configured to display the matching accuracy and the synthesis image. The medical image processing apparatus captures and synthesizes a plurality of images and display information of matching accuracy of the plurality of images. The medical image processing apparatus may provide a more accurate image by correcting a mismatched region of an overlapped region between the images.

### CROSS-REFERENCE TO RELATED APPLICATION(S)

The present application claims priority under 35 U.S.C. §365 to International Patent Application No. PCT/KR2016/001804 filed Feb. 24, 2016, which claims priority to Korean Patent Application Nos. 10-2015-0025909, filed Feb. 24, 2015 and 10-2016-0018544, filed Feb. 17, 2016, each of which are incorporated herein by reference into the present disclosure as if fully set forth herein.

### TECHNICAL FIELD

The present disclosure relates to medical image processing apparatuses and medical image processing methods, and more particularly, to a medical image processing apparatus and a medical image processing method for displaying image information and correcting an image.

### BACKGROUND

In general, X-rays are electromagnetic waves having a wavelength of 0.01 to 100 Å and can pass through an object. Thus, they may be commonly used in a wide range of applications, such as medical equipment that take images of the inside of a living body and non-destructive testing equipment for industrial use.

X-ray imaging apparatuses using X-rays allow X-rays emitted by an X-ray source to pass through an object, and detect a difference between the intensities of the passed X-rays from an X-ray detector to thereby acquire an X-ray image of the object. X-ray imaging apparatuses also easily identify the internal structure of an object based on an X-ray image of the object and diagnose a disease of the object.

### SUMMARY

According to an aspect of exemplary embodiment, there is provided a medical image processing apparatus comprising: a processor configured to acquire a first image and a second image captured by radiating an X ray to an object, generate a synthesis image by overlapping a first region of the first image with a second region of the second image, and determine matching accuracy representing a degree to which the first region and the second region match with each other; and a display configured to display the matching accuracy and the synthesis image.

According to an aspect of exemplary embodiment, there is provided a An X-ray imaging apparatus comprising: a source configured to radiate an X-ray to an object; a detector configured to detect an X-ray transmitted by the object; a processor configured to control a location of at least one of the source and the detector, acquire an image captured based on the location of the source and the location of the detector, and acquire error information of the captured image due to a location error of at least one of the source and the detector from the captured image; and a display configured to display information about correction of the at least one location error, based on the captured image and the error information of the captured image.

Additional aspects will be set forth in part in the description which follows and, in part, will be apparent from the description, or may be learned by practice of the presented embodiments.

Medical image processing apparatuses capture and synthesize a plurality of images and display information of matching accuracy of the plurality of images. The medical image processing apparatuses may provide a more accurate image by correcting a mismatched region of an overlapped region between the images.

X-ray imaging apparatuses allow for correction of a location error thereof via a user interface (UI) screen image, simplify a correction procedure, and enhance the quality of image.

### BRIEF DESCRIPTION OF THE DRAWINGS

These and/or other aspects will become apparent and more readily appreciated from the following description of the embodiments, taken in conjunction with the accompanying drawings in which:

FIG. 1 is a schematic diagram of an X-ray system according to an embodiment;

FIG. 2 is a perspective view of a fixed type X-ray apparatus according to an embodiment;

FIG. 3 is a schematic diagram of a mobile X-ray apparatus according to an embodiment;

FIG. 4 is a schematic diagram showing a detailed configuration of a detector according to an embodiment;

FIG. 5 explains a result of synthesizing a plurality of images having an overlapped region, according to an embodiment;

FIG. 6A is a block diagram of a structure of a medical image processing apparatus according to an embodiment;

FIG. 6B is a block diagram of a structure of a medical image processing apparatus according to another embodiment;

FIG. 7A is a flowchart of a medical image processing method according to an embodiment;

FIG. 7B is a flowchart of a medical image processing method according to another embodiment;

FIG. 8 explains a method of synthesizing a plurality of images captured according to locations on an object, according to an embodiment;

FIG. 9A explains a screen image that is output to a medical image processing apparatus and displays matching accuracy, according to an embodiment;

FIG. 9B explains a screen image that is output to a medical image processing apparatus and displays matching accuracy, according to another embodiment;

FIG. 10A explains a user interface (UI) screen image via which a medical image processing apparatus corrects a synthesis image, according to an embodiment;

FIG. 10B explains a UI screen image via which a medical image processing apparatus corrects a synthesis image, according to another embodiment;

FIG. 11 explains an operation of an X-ray imaging apparatus according to an embodiment;

FIG. 12A is a block diagram of a structure of an X-ray imaging apparatus according to an embodiment;

FIG. 12B is a block diagram of a structure of an X-ray imaging apparatus according to another embodiment;

FIG. 13 is a flowchart of a method of operating an X-ray imaging apparatus, according to an embodiment;

FIG. 14 explains a structure of an X-ray imaging apparatus according to an embodiment;

FIG. 15A explains a photographing operation of an X-ray imaging apparatus according to an embodiment;

FIG. 15B illustrates a synthesis image generated by an X-ray imaging apparatus;

FIG. 16A explains a photographing operation of an X-ray imaging apparatus according to an embodiment;

FIG. 16B illustrates a synthesis image generated by an X-ray imaging apparatus;

FIG. 17A is a flowchart of a method of operating an X-ray imaging apparatus, according to another embodiment;

FIG. 17B is a flowchart of a method of operating an X-ray imaging apparatus, according to another embodiment;

FIG. 18A explains a method of correcting a location error by using a plurality of captured images via a UI screen image, according to an embodiment;

FIG. 18B explains a method of correcting a location error by using a plurality of captured images via a UI screen image, according to another embodiment;

FIG. 19 explains a synthesis image in which location error correction of an X-ray imaging apparatus has been reflected, and a synthesis image in which location error correction of an X-ray imaging apparatus has not yet been reflected, according to an embodiment;

FIG. 20A explains a method of correcting a location error by using a plurality of captured images via a UI screen image, according to another embodiment;

FIG. 20B explains a synthesis image in which location error correction of an X-ray imaging apparatus has been reflected, and a synthesis image in which location error correction of an X-ray imaging apparatus has not yet been reflected, according to an embodiment;

FIG. 21 is a flowchart of a method of operating an X-ray imaging apparatus, according to an embodiment;

FIGS. 22A and 22B explain a method of correcting a location error by using a captured image via a UI screen image, according to an embodiment;

FIG. 23 is a flowchart of a method of operating an X-ray imaging apparatus, according to an embodiment; and

FIG. 24 explains a method of correcting a location error by using a captured image via a UI screen image, according to an embodiment.

### DETAILED DESCRIPTION

According to an aspect of exemplary embodiment, there is provided a medical image processing apparatus comprising: a processor configured to acquire a first image and a second image captured by radiating an X ray to an object, generate a synthesis image by overlapping a first region of the first image with a second region of the second image, and determine matching accuracy representing a degree to which the first region and the second region match with each other; and a display configured to display the matching accuracy and the synthesis image.

According to an aspect of exemplary embodiment, there is provided a An X-ray imaging apparatus comprising: a source configured to radiate an X-ray to an object; a detector configured to detect an X-ray transmitted by the object; a processor configured to control a location of at least one of the source and the detector, acquire an image captured based on the location of the source and the location of the detector, and acquire error information of the captured image due to a location error of at least one of the source and the detector from the captured image; and a display configured to display information about correction of the at least one location error, based on the captured image and the error information of the captured image.

A medical image processing apparatus comprises a processor configured to acquire a first image and a second image captured by radiating an X ray to an object, generate a synthesis image by overlapping a first region of the first image with a second region of the second image, and determine matching accuracy representing a degree to which the first region and the second region match with each other; and a display configured to display the matching accuracy and the synthesis image.

The display displays at least one of information about a length of an overlapped region between the first image and the second image on the synthesis image and information about a location of the overlapped region on the synthesis image.

The display displays, on the synthesis image, a marker indicating a location of an overlapped region between the first image and the second image.

The display displays information representing whether matching between the first image and the second image has succeeded, based on the matching accuracy.

When the first region of the first image and the second region of the second image do not match with each other, the display distinguishably displays a predetermined portion corresponding to a mismatched region between the first region of the first image and the second region of the second image.

The display displays, together with the synthesis image, a marker indicating a location of an overlapped region between the first image and the second image, and the processor distinguishably sets at least one of a color, shape, and pattern of the marker, based on the matching accuracy.

A medical image processing apparatus further comprises an input unit configured to receive a user input for correcting a range of at least one of the first region of the first image and the second region of the second image, wherein the processor corrects the range of the at least one of the first region and the second region, based on the user input, and re-generates a synthesis image by using a result of the correction.

The user input comprises at least one of an input of correcting a section of the overlapped region and an input of adjusting a magnification ratio of the first image or the second image.

The processor receives first location information representing a location of a detector for detecting an X-ray transmitted by the object during capturing of the first image, and receives second location information representing a location of the detector during capturing of the second image, determines the first region of the first image and the second region of the second image based on the first location information and the second location information, and overlaps the first region with the second region to generate the synthesis image.

A medical image processing method comprises acquiring a first image and a second image captured by radiating an X ray to an object; generating a synthesis image by overlapping a first region of the first image with a second region of the second image; determining matching accuracy representing a degree to which the first region and the second region match with each other; and displaying the matching accuracy and the synthesis image.

The displaying of the matching accuracy information and the synthesis image comprises: displaying, together with the synthesis image, a marker indicating a location of an overlapped region between the first image and the second image; and distinguishably setting at least one of a color, shape, and pattern of the marker, based on the matching accuracy.

When the first region of the first image and the second region of the second image do not match with each other, the displaying of the matching accuracy information and the synthesis image comprises at least one of: distinguishably displaying a predetermined portion corresponding to a mismatched region between the first region of the first image and the second region of the second image; and changing and displaying at least one of a color, shape, and pattern of a marker representing a location of an overlapped region between the first image and the second image.

The medical image processing method of claim 10, further comprises receiving a user input for correcting a range of at least one of the first region of the first image and the second region of the second image; and correcting the range of the at least one of the first region and the second region, based on the user input, and re-generating a synthesis image by using a result of the correction.

The generating of the synthesis image comprises: receiving first location information representing a location of a detector for detecting an X-ray transmitted by the object during capturing of the first image, and receives second location information representing a location of the detector during capturing of the second image; and determining the first region of the first image and the second region of the second image based on the first location information and the second location information, and overlapping the first region with the second region to generate the synthesis image.

An X-ray imaging apparatus comprises a source configured to radiate an X-ray to an object; a detector configured to detect an X-ray transmitted by the object; a processor configured to control a location of at least one of the source and the detector, acquire an image captured based on the location of the source and the location of the detector, and acquire error information of the captured image due to a location error of at least one of the source and the detector from the captured image; and a display configured to display information about correction of the at least one location error, based on the captured image and the error information of the captured image.

The processor generates a predicted image based on the location of the source and the location of the detector, compares the captured image with the predicted image, and acquires the error information of the captured image according to a result of the comparison.

The X-ray imaging apparatus further comprises an input unit configured to receive a user input of correcting the at least one location error, wherein the processor corrects the at least one location error by changing a location of at least one of the source and the detector, based on the user input.

The processor acquires a first image and a second image of the object captured based on the correction of the at least one location error, and overlaps a first region of the first image with a second region of the second image, wherein the first and second regions correspond to a predetermined region of the object, to generate a synthesis image, and the display displays the synthesis image.

The processor detects an error due to a difference between magnification ratios of the captured image and the predicted image and corrects the magnification ratio of the captured image to the magnification ratio of the predicted image based on the user input, and the input unit receives a user input for correcting the error due to the difference between the magnification ratios.

The X-ray imaging apparatus further comprises a memory configured to store a driving range and location information of the X-ray imaging apparatus, wherein the processor controls a photographing operation based on the driving range and the location information and acquires a corrected driving range and corrected location information based on the location error of the at least one of the source and the detector, and the memory stores the corrected driving range and the corrected location information.

The processor detects a collimator region corresponding to an X-ray irradiated region of a collimator from the captured image, acquires information about an area of the collimator region and a central point of the collimator region from the captured image, and compares the information about the area of the collimator region and the central point of the collimator region with preset information about the area of the collimator region and a central point of the detector to acquire the error information of the captured image.

The input unit receives at least one of a user input of adjusting the area of the collimator region and a user input of adjusting the central point of the collimator region, and the processor adjusts at least one of the area of the collimator region and the central point of the collimator region according to the received user input.

The processor detects a collimator region corresponding to an X-ray irradiated region of a collimator from the captured image, acquires, from the captured image, first captured coordinate values representing coordinate values of a plurality of vertices of the collimator region, calculates first predicted coordinate values representing predicted coordinate values of the plurality of vertices of the collimator region, based on the location of the source and the location of the detector, and acquires error information of the captured image with respect to the collimator region by comparing the first predicted coordinate values with the first captured coordinate values, and the display displays the error information with respect to the collimator region.

The input unit receives a user input of moving the collimator region so that the first captured coordinate values are identical with the first predicted coordinate values, based on the error information, and the processor controls the source to move the collimator region based on the user input.

A method of operating an X-ray imaging apparatus, the method comprises controlling a location of at least one of a source that radiates an X ray to an object and a detector that detects an X ray transmitted by the object; acquiring an image captured based on a location of the source and a location of the detector; acquiring error information of the captured image due to a location error of at least one of the source and the detector from the captured image; and displaying information about correction of the at least one location error, based on the captured image and the error information of the captured image.

The acquiring of the error information of the captured image comprises: generating a predicted image based on the location of the source and the location of the detector; and comparing the captured image with the predicted image and acquiring the error information of the captured image according to a result of the comparison.

The method further comprises receiving a user input of correcting the at least one location error from a user interface (UI) screen image; and correcting the at least one location error by changing the location of the at least one of the source and the detector, based on the user input.

The method further comprises acquiring a first image and a second image of the object, based on the correction of the at least one location error; generating a synthesis image by overlapping a first region of the first image with a second region of the second image, wherein the first and second regions correspond to a predetermined region of the object; and displaying the synthesis image.

The acquiring of the error information of the captured image due to the location error of the at least one of the source and the detector comprises detecting an error due to a difference between magnification ratios of the captured image and the predicted image, the receiving of the user input of correcting the at least one location error comprises receiving a user input for correcting the error due to the difference between the magnification ratios, and

the correcting of the at least one location error by changing the location of the at least one of the source and the detector comprises correcting the magnification ratio of the captured image to the magnification ratio of the predicted image, based on the user input of correcting the error due to the difference between the magnification ratios.

The method further comprises storing a driving range and location information of the X-ray imaging apparatus, wherein the acquiring of the image captured based on the location of the source and the location of the detector comprises controlling a photographing operation based on the driving range and the location information, and the method further comprises: acquiring a corrected driving range and corrected location information based on the location error of the at least one of the source and the detector; and storing the corrected driving range and the corrected location information.

The acquiring of the error information of the captured image comprises: detecting a collimator region corresponding to an X-ray irradiated region of a collimator from the captured image; acquiring information about an area of the collimator region and a central point of the collimator region from the captured image; and comparing the information about the area of the collimator region and the central point of the collimator region with preset information about the area of the collimator region and a central point of the detector.

The receiving of the user input of correcting the at least one location error from the UI screen image comprises: receiving at least one of a user input of adjusting the area of the collimator region and a user input of adjusting the central point of the collimator region; and adjusting at least one of the area of the collimator region and the central point of the collimator region according to the received user input.

The acquiring of the error information of the captured image comprises: detecting a collimator region corresponding to an X-ray irradiated region of a collimator from the captured image; acquiring, from the captured image, first captured coordinate values representing coordinate values of a plurality of vertices of the collimator region; calculating first predicted coordinate values representing predicted coordinate values of the plurality of vertices of the collimator region, based on the location of the source and the location of the detector; and acquiring error information of the captured image by comparing the first predicted coordinate values with the first captured coordinate values, and the displaying of the UI screen image comprises displaying error information with respect to the collimator region.

The receiving of the user input of correcting the at least one location error from the UI screen image comprises receiving a user input of moving the collimator region so that the first captured coordinate values are identical with the first predicted coordinate values, based on the error information, and the correcting of the at least one location error by changing the location of the at least one of the source and the detector comprises controlling the source to move the collimator region according to a user input of moving the collimator region so that the first captured coordinate values are identical with the first predicted coordinate values.

Advantages and features of one or more embodiments of the present invention and methods of accomplishing the same may be understood more readily by reference to the following detailed description of the embodiments and the accompanying drawings. In this regard, the present embodiments may have different forms and should not be construed as being limited to the descriptions set forth herein. Rather, these embodiments are provided so that this disclosure will be thorough and complete and will fully convey the concept of the present embodiments to one of ordinary skill in the art, and the present invention will only be defined by the appended claims.

Hereinafter, the terms used in the specification will be briefly described, and then the present disclosure will be described in detail.

The terms used in this specification are those general terms currently widely used in the art in consideration of functions regarding the inventive concept, but the terms may vary according to the intention of those of ordinary skill in the art, precedents, or new technology in the art. Also, some terms may be arbitrarily selected by the applicant, and in this case, the meaning of the selected terms will be described in detail in the detailed description of the present specification. Thus, the terms used herein have to be defined based on the meaning of the terms together with the description throughout the specification.

Throughout the specification, an “image” may denote multi-dimensional data composed of discrete image elements (for example, pixels in a two-dimensional image and voxels in a three-dimensional image). For example, an image may be a medical image of an object acquired by an X-ray apparatus, a computed tomography (CT) apparatus, a magnetic resonance imaging (MM) apparatus, an ultrasound diagnosis apparatus, or another medical imaging apparatus.

Furthermore, in the present specification, an “object” may be a human, an animal, or a part of a human or animal. For example, the object may include an organ (for example, the liver, the heart, the womb, the brain, breasts, or the abdomen), blood vessels, or a combination thereof. The object may be a phantom. The phantom denotes a material having a volume, a density, and an effective atomic number that are approximately the same as those of a living organism. For example, the phantom may be a spherical phantom having similar properties to those of the human body.

Throughout the specification, a “user” may be, but is not limited to, a medical expert, for example, a medical doctor, a nurse, a medical laboratory technologist, or a medical imaging expert, or a technician who repairs medical apparatuses.

An X-ray apparatus is a medical imaging apparatus that acquires images of internal structures of an object by transmitting an X-ray through the human body. The X-ray apparatus may acquire medical images of an object more simply within a shorter time than other medical imaging apparatuses including an Mill apparatus and a CT apparatus. Therefore, the X-ray apparatus is widely used in simple chest imaging, simple abdomen imaging, simple skeleton imaging, simple nasal sinuses imaging, simple neck soft tissue imaging, and breast imaging.

While such terms as “first,” “second,” etc., may be used to describe various components, such components must not be limited to the above terms. The above terms are used only to distinguish one component from another. For example, a first component discussed below could be termed a second component, and similarly, a second component may be termed a first component without departing from the teachings of this disclosure. The term “and/or” includes any and all combinations of one or more of the associated listed items.

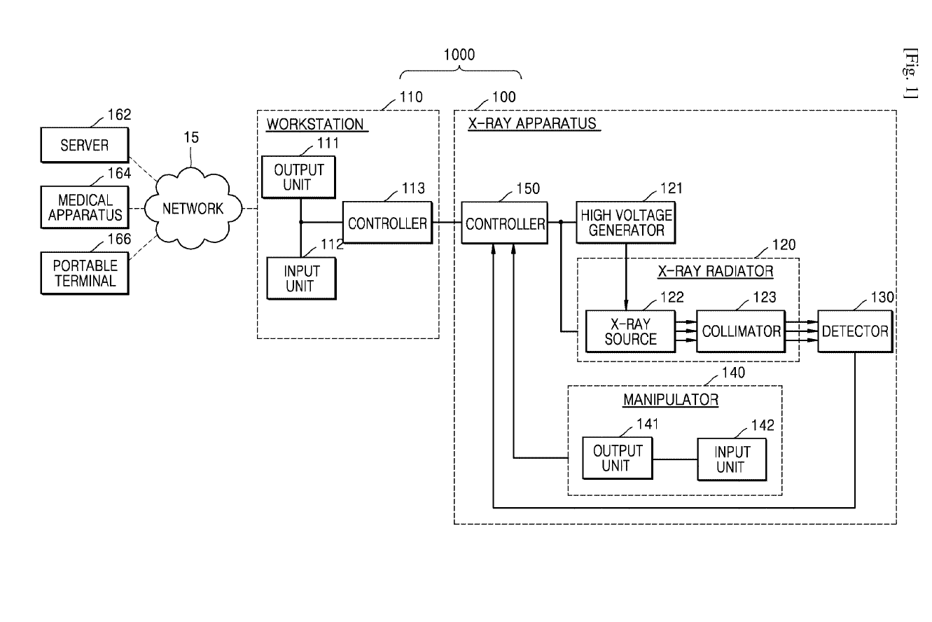

FIG. 1 is a block diagram of an X-ray system 1000.

Referring to FIG. 1, the X-ray system 1000 includes an X-ray apparatus 100 and a workstation 110. The X-ray apparatus 100 shown in FIG. 1 may be a fixed-type X-ray apparatus or a mobile X-ray apparatus. The X-ray apparatus 100 may include an X-ray radiator 120, a high voltage generator 121, a detector 130, a manipulator 140, and a controller 150. The controller 150 may control overall operations of the X-ray apparatus 100.

The high voltage generator 121 generates a high voltage for generating X-rays, and applies the high voltage to an X-ray source 122.

The X-ray radiator 120 includes the X-ray source 122 receiving the high voltage from the high voltage generator 121 to generate and radiate X-rays, and a collimator 123 for guiding a path of the X-ray radiated from the X-ray source 122 and adjusting an irradiation region radiated by the X-ray.

The X-ray source 122 includes an X-ray tube that may be realized as a vacuum tube diode including a cathode and an anode. An inside of the X-ray tube is set as a high vacuum state of about 10 mmHg, and a filament of the anode is heated to a high temperature to generate thermal electrons. The filament may be a tungsten filament, and a voltage of about 10V and a current of about 3 to 5 A may be applied to an electric wire connected to the filament to heat the filament.

In addition, when a high voltage of about 10 to about 300 kVp is applied between the cathode and the anode, the thermal electrons are accelerated to collide with a target material of the cathode, and then, an X-ray is generated. The X-ray is radiated outside via a window, and the window may be formed of a beryllium thin film. In this case, most of the energy of the electrons colliding with the target material is consumed as heat, and remaining energy is converted into the X-ray.

The cathode is mainly formed of copper, and the target material is disposed opposite to the anode. The target material may be a high resistive material such as chromium (Cr), iron (Fe), cobalt (Co), nickel (Ni), tungsten (W), or molybdenum (Mo). The target material may be rotated by a rotating field. When the target material is rotated, an electron impact area is increased, and a heat accumulation rate per unit area may be increased to be at least ten times greater than that of a case where the target material is fixed.

The voltage applied between the cathode and the anode of the X-ray tube is referred to as a tube voltage, and the tube voltage is applied from the high voltage generator 121 and a magnitude of the tube voltage may be expressed by a crest value (kVp). When the tube voltage increases, a velocity of the thermal electrons increases, and accordingly, an energy of the X-ray (energy of photon) that is generated when the thermal electrons collide with the target material is increased. The current flowing in the X-ray tube is referred to as a tube current that may be expressed as an average value (mA). When the tube current increases, the number of thermal electrons emitted from the filament is increased, and accordingly, the X-ray dose (the number of X-ray photons) generated when the thermal electrons collide with the target material is increased.

Therefore, the energy of the X-ray may be adjusted according to the tube voltage, and the intensity of the X-ray or the X-ray dose may be adjusted according to the tube current and the X-ray exposure time.

The detector 130 detects an X-ray that is radiated from the X-ray radiator 120 and has been transmitted through an object. The detector 130 may be a digital detector. The detector 130 may be implemented by using a thin film transistor (TFT) or a charge coupled device (CCD). Although the detector 130 is included in the X-ray apparatus 100 in FIG. 1, the detector 130 may be an X-ray detector that is a separate device capable of being connected to or separated from the X-ray apparatus 100.

The X-ray apparatus 100 may further include a manipulator 140 for providing a user with an interface for manipulating the X-ray apparatus 100. The manipulator 140 may include an output unit 141 and an input unit 142. The input unit 142 may receive from a user a command for manipulating the X-ray apparatus 100 and various types of information related to X-ray imaging. The controller 150 may control or manipulate the X-ray apparatus 100 according to the information received by the input unit 142. The output unit 141 may output sound representing information related to an imaging operation such as the X-ray radiation under the control of the controller 150.

The workstation 110 and the X-ray apparatus 100 may be connected to each other by wire or wirelessly. When they are connected to each other wirelessly, a device (not shown) for synchronizing clock signals with each other may be further included. The workstation 110 and the X-ray apparatus 100 may exist within physically separate spaces.

The workstation 110 may include an output unit 111, an input unit 112, and a controller 113. The output unit 111 and the input unit 112 provide a user with an interface for manipulating the workstation 110 and the X-ray apparatus 200. The controller 113 may control the workstation 110 and the X-ray apparatus 200.

The X-ray apparatus 100 may be controlled via the workstation 110 or may be controlled by the controller 150 included in the X-ray apparatus 100. Accordingly, a user may control the X-ray apparatus 100 via the workstation 110 or may control the X-ray apparatus 100 via the manipulator 140 and the controller 150 included in the X-ray apparatus 100. In other words, a user may remotely control the X-ray apparatus 100 via the workstation 110 or may directly control the X-ray apparatus 100.

Although the controller 113 of the workstation 110 is separate from the controller 150 of the X-ray apparatus 100 in FIG. 1, FIG. 1 is only an example. As another example, the controllers 113 and 150 may be integrated into a single controller, and the single controller may be included in only one of the workstation 110 and the X-ray apparatus 100. Hereinafter, the controllers 113 and 150 may denote the controller 113 of the workstation 110 and/or the controller 150 of the X-ray apparatus 100.

The output unit 111 and the input unit 112 of the workstation 110 may provide a user with an interface for manipulating the X-ray apparatus 100, and the output unit 141 and the input unit 142 of the X-ray apparatus 100 may also provide a user with an interface for manipulating the X-ray apparatus 100. Although the workstation 110 and the X-ray radiation apparatus 100 include the output units 111 and 141, respectively, and the input units 112 and 142, respectively, in FIG. 1, embodiments are not limited thereto. Only one of the workstation 110 and the X-ray apparatus 100 may include an output unit or an input unit.

Hereinafter, the input units 112 and 142 may denote the input unit 112 of the workstation 110 and/or the input unit 142 of the X-ray apparatus 100, and the output units 111 and 141 may denote the output unit 111 of the workstation 110 and/or the output unit 141 of the X-ray apparatus 100.

Examples of the input units 112 and 142 may include a keyboard, a mouse, a touch screen, a voice recognizer, a fingerprint recognizer, an iris recognizer, and other input devices which are well known to one of ordinary skill in the art. The user may input a command for radiating the X-ray via the input units 112 and 142, and the input units 112 and 142 may include a switch for inputting the command. The switch may be configured so that a radiation command for radiating the X-ray may be input only when the switch is pushed in two steps.

In other words, when the user pushes the switch, a prepare command for performing a pre-heating operation for X-ray radiation may be input, and in this state, when the user pushes the switch deeper, a radiation command for performing substantial X-ray radiation may be input. When the user manipulates the switch as described above, the controllers 113 and 150 generate signals corresponding to the commands input through the switch manipulation, that is, a prepare signal, and transmit the generated signals to the high voltage generator 121 generating a high voltage for generating the X-ray.

When the high voltage generator 121 receives the prepare signal from the controllers 113 and 150, the high voltage generator 121 starts a pre-heating operation, and when the pre-heating is finished, the high voltage generator 121 outputs a ready signal to the controllers 113 and 150. In addition, the detector 130 also needs to prepare to detect the X-ray, and thus the high voltage generator 121 performs the pre-heating operation and the controllers 113 and 150 transmit a prepare signal to the detector 130 so that the detector 130 may prepare to detect the X-ray transmitted through the object. The detector 130 prepares to detect the X-ray in response to the prepare signal, and when the preparing for the detection is finished, the detector 130 outputs a ready signal to the controllers 113 and 150.

When the pre-heating operation of the high voltage generator 121 is finished and the detector 130 is ready to detect the X-ray, the controllers 113 and 150 transmit a radiation signal to the high voltage generator 121, the high voltage generator 121 generates and applies the high voltage to the X-ray source 122, and the X-ray source 122 radiates the X-ray.

When the controllers 113 and 150 transmit the radiation signal to the high voltage generator 121, the controllers 113 and 150 may transmit a sound output signal to the output units 111 and 141 so that the output units 111 and 141 output a predetermined sound and the object may recognize the radiation of the X-ray. The output units 111 and 141 may also output a sound representing information related to photographing in addition to the X-ray radiation. In FIG. 1, the output unit 141 is included in the manipulator 140; however, the embodiments are not limited thereto, and the output unit 141 or a portion of the output unit 141 may be located elsewhere. For example, the output unit 141 may be located on a wall of an examination room in which the X-ray photographing of the object is performed.

The controllers 113 and 150 control locations of the X-ray radiator 120 and the detector 130, photographing timing, and photographing conditions, according to photographing conditions set by the user.

In more detail, the controllers 113 and 150 control the high voltage generator 121 and the detector 130 according to the command input via the input units 112 and 142 so as to control radiation timing of the X-ray, an intensity of the X-ray, and a region radiated by the X-ray. In addition, the control units 113 and 150 adjust the location of the detector 130 according to a predetermined photographing condition, and controls operation timing of the detector 130.

Furthermore, the controllers 113 and 150 generate a medical image of the object by using image data received via the detector 130. In detail, the controllers 113 and 150 may receive the image data from the detector 130, and then, generate the medical image of the object by removing noise from the image data and adjusting a dynamic range and interleaving of the image data.

The output units 111 and 141 may output the medical image generated by the controllers 113 and 150. The output units 111 and 141 may output information that is necessary for the user to manipulate the X-ray apparatus 100, for example, a user interface (UI), user information, or object information. Examples of the output units 111 and 141 may include a speaker, a printer, a cathode ray tube (CRT) display, a liquid crystal display (LCD), a plasma display panel (PDP), an organic light emitting diode (OLED) display, a field emission display (FED), a light emitting diode (LED) display, a vacuum fluorescent display (VFD), a digital light processing (DLP) display, a flat panel display (FPD), a three-dimensional 3D) display, a transparent display, and other various output devices well known to one of ordinary skill in the art.

The maximum depth shown in FIG. 1 may further include a communicator (not shown) that may be connected to a server 162, a medical apparatus 164, and a portable terminal 166 via a network 15.

The communicator may be connected to the network 15 by wire or wirelessly to communicate with the server 162, the medical apparatus 164, or the portable terminal 166. The communicator may transmit or receive data related to diagnosis of the object via the network 15, and may also transmit or receive medical images captured by the medical apparatus 164, for example, a CT apparatus, an MRI apparatus, or an X-ray apparatus. Moreover, the communicator may receive a medical history or treatment schedule of an object (e.g., a patient) from the server 162 to diagnose a disease of the object. Also, the communicator may perform data communication with the portable terminal 166 such as a mobile phone, a personal digital assistant (PDA), or a laptop computer of a medical doctor or a client, as well as the server 162 or the medical apparatus 164 in a hospital.

The communicator may include one or more elements enabling communication with external apparatuses. For example, the communicator may include a local area communication module, a wired communication module, and a wireless communication module.

The local area communication module refers to a module for performing local area communication with an apparatus located within a predetermined distance. Examples of local area communication technology may include, but are not limited to, a wireless local area network (LAN), Wi-Fi, Bluetooth, ZigBee, Wi-Fi Direct (WFD), ultra wideband (UWD), infrared data association (IrDA), Bluetooth low energy (BLE), and near field communication (NFC).

The wired communication module refers to a module for communicating by using an electric signal or an optical signal. Examples of wired communication technology may include wired communication techniques using a pair cable, a coaxial cable, and an optical fiber cable, and other wired communication techniques that are well known to one of ordinary skill in the art.

The wireless communication module transmits and receives a wireless signal to and from at least one selected from a base station, an external apparatus, and a server in a mobile communication network. Here, examples of the wireless signal may include a voice call signal, a video call signal, and various types of data according to text/multimedia messages transmission.

The X-ray apparatus 100 shown in FIG. 1 may include a plurality of digital signal processors (DSPs), an ultra-small calculator, and a processing circuit for special purposes (for example, high speed analog/digital (A/D) conversion, high speed Fourier transformation, and an array process).

In addition, communication between the workstation 110 and the X-ray apparatus 100 may be performed using a high speed digital interface, such as low voltage differential signalling (LVDS), asynchronous serial communication, such as a universal asynchronous receiver transmitter (UART), a low latency network protocol, such as error synchronous serial communication or a controller area network (CAN), or any of other various communication methods that are well known to one of ordinary skill in the art.

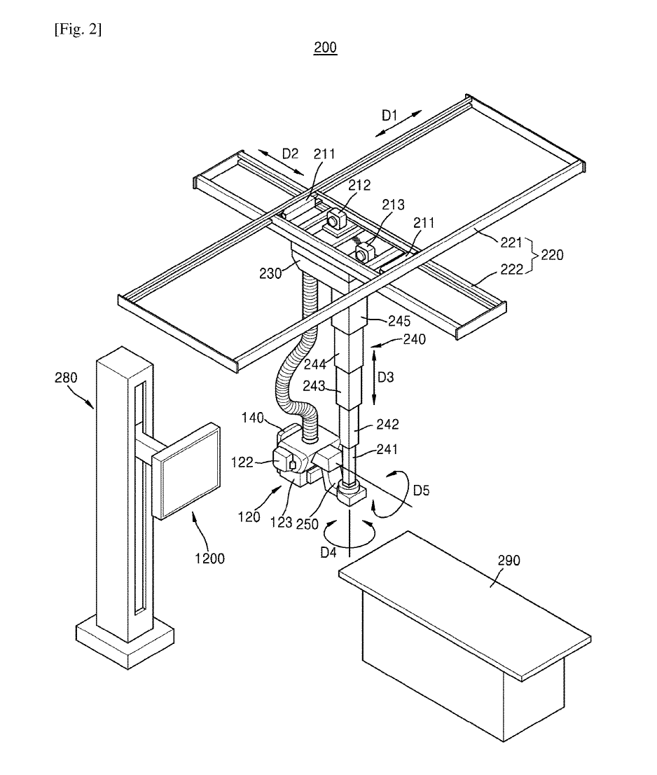

FIG. 2 is a perspective view of a fixed type X-ray apparatus 200. The fixed type X-ray apparatus 200 may be another embodiment of the X-ray apparatus 100 of FIG. 1. Components included in the fixed type X-ray apparatus 200 that are the same as those of the X-ray apparatus 100 of FIG. 1 use the same reference numerals, and repeated descriptions thereof will be omitted.

Referring to FIG. 2, the fixed type X-ray apparatus 200 includes a manipulator 140 providing a user with an interface for manipulating the X-ray apparatus 200, an X-ray radiator 120 radiating an X-ray to an object, a detector 130 detecting an X-ray that has passed through the object, first, second, and third motors 211, 212, and 213 providing a driving power to transport the X-ray radiator 120, a guide rail 220, a moving carriage 230, and a post frame 240. The guide rail 220, the moving carriage 230, and the post frame 240 are formed to transport the X-ray radiator 120 by using the driving power of the first, second, and third motors 211, 212, and 213.

The guide rail 220 includes a first guide rail 221 and a second guide rail 222 that are provided to form a predetermined angle with respect to each other. The first guide rail 221 and the second guide rail 222 may respectively extend in directions crossing each other at 90°.

The first guide rail 221 is provided on the ceiling of an examination room in which the X-ray apparatus 200 is disposed.

The second guide rail 222 is located under the first guide rail 221, and is mounted so as to slide along the first guide rail 221. A roller (not shown) that may move along the first guide rail 221 may be provided on the first guide rail 221. The second guide rail 222 is connected to the roller to move along the first guide rail 221.

A first direction D1 is defined as a direction in which the first guide rail 221 extends, and a second direction D2 is defined as a direction in which the second guide rail 222 extends. Therefore, the first direction D1 and the second direction D2 cross each other at 90°, and may be parallel to the ceiling of the examination room.

The moving carriage 230 is disposed under the second guide rail 222 so as to move along the second guide rail 222. A roller (not shown) moving along the second guide rail 222 may be provided on the moving carriage 230.

Therefore, the moving carriage 230 may move in the first direction D1 together with the second guide rail 222, and may move in the second direction D2 along the second guide rail 222.

The post frame 240 is fixed on the moving carriage 230 and located under the moving carriage 230. The post frame 240 may include a plurality of posts 241, 242, 243, 244, and 245.

The plurality of posts 241, 242, 243, 244, and 245 are connected to each other to be foldable, and thus, the post frame 240 may have a length that is adjustable in a vertical direction of the examination room while in a state of being fixed to the moving carriage 230.

A third direction D3 is defined as a direction in which the length of the post frame 240 increases or decreases. Therefore, the third direction D3 may be perpendicular to the first direction D1 and the second direction D2.

The detector 130 detects the X-ray that has passed through the object, and may be combined with a table type receptor 290 or a stand type receptor 280.

A rotating joint 250 is disposed between the X-ray radiator 120 and the post frame 240. The rotating joint 250 allows the X-ray radiator 120 to be coupled to the post frame 240, and supports a load applied to the X-ray radiator 120.

The X-ray radiator 120 connected to the rotating joint 250 may rotate on a plane that is perpendicular to the third direction D3. In this case, a rotating direction of the X-ray radiator 120 may be defined as a fourth direction D4.

Also, the X-ray radiator 120 may be configured to be rotatable on a plane perpendicular to the ceiling of the examination room. Therefore, the X-ray radiator 120 may rotate in a fifth direction D5 that is a rotating direction about an axis that is parallel with the first direction D1 or the second direction D2, with respect to the rotating joint 250.

The first, second, and third motors 211, 212, and 213 may be provided to move the X-ray radiator 120 in the first, second, and third directions D1, D2, and D3. The first, second, and third motors 211, 212, and 213 may be electrically driven, and the first, second, and third motors 211, 212, and 213 may respectively include an encoder.

The first, second, and third motors 211, 212, and 213 may be disposed at various locations in consideration of design convenience. For example, the first motor 211, moving the second guide rail 222 in the first direction D1, may be disposed around the first guide rail 221, the second motor 212, moving the moving carriage 230 in the second direction D2, may be disposed around the second guide rail 222, and the third motor 213, increasing or reducing the length of the post frame 240 in the third direction D3, may be disposed in the moving carriage 230. In another example, the first, second, and third motors 211, 212, and 213 may be connected to a driving power transfer unit (not shown) so as to linearly move the X-ray radiator 120 in the first, second, and third directions D1, D2, and D3. The driving power transfer unit may be a combination of a belt and a pulley, a combination of a chain and a sprocket, or a shaft, which are generally used.

In another example, motors (not shown) may be disposed between the rotating joint 250 and the post frame 240 and between the rotating joint 250 and the X-ray radiator 120 in order to rotate the X-ray radiator 120 in the fourth and fifth directions D4 and D5.

The manipulator 140 may be disposed on a side surface of the X-ray radiator 120.

FIG. 2 shows the fixed type X-ray apparatus 200 connected to the ceiling of the examination room, the fixed type X-ray apparatus 200 is merely an example for convenience of comprehension. That is, X-ray apparatuses according to embodiments of the present disclosure may include X-ray apparatuses having various structures that are well known to one of ordinary skill in the art, for example, a C-arm-type X-ray apparatus and an angiography X-ray apparatus, in addition to the fixed type X-ray apparatus 200 of FIG. 2.

FIG. 3 is a diagram showing a configuration of a mobile X-ray apparatus 300 capable of performing an X-ray photographing operation regardless of a place where the photographing operation is performed. The mobile X-ray apparatus 300 may be another embodiment of the X-ray apparatus 100 of FIG. 1. Components included in the mobile X-ray apparatus 300 that are the same as those of the X-ray apparatus 100 of FIG. 1 use the same reference numerals as those used in FIG. 1, and a repeated description thereof will be omitted.

Referring to FIG. 3, the mobile X-ray apparatus 300 includes a transport unit 370 including a wheel for transporting the mobile X-ray apparatus 300, a main unit 305, an X-ray radiator 120, and a detector 130 detecting an X-ray that is radiated from the X-ray radiator 120 toward an object and transmitted through the object. The main unit 305 includes a manipulator 140 providing a user with an interface for manipulating the mobile X-ray apparatus 300, a high voltage generator 121 generating a high voltage applied to an X-ray source 122, and a controller 150 controlling overall operations of the mobile X-ray apparatus 300. The X-ray radiator 120 includes the X-ray source 122 generating the X-ray, and a collimator 123 guiding a path along which the generated X-ray is emitted from the X-ray source 122 and adjusting an irradiation region radiated by the X-ray.

The detector 130 in FIG. 3 may not be combined with any receptor, and the detector 130 may be a portable detector which can exist anywhere.

In FIG. 3, the manipulator 140 is included in the main unit 305; however, embodiments are not limited thereto. For example, as illustrated in FIG. 2, the manipulator 140 of the mobile X-ray apparatus 300 may be disposed on a side surface of the X-ray radiator 120.

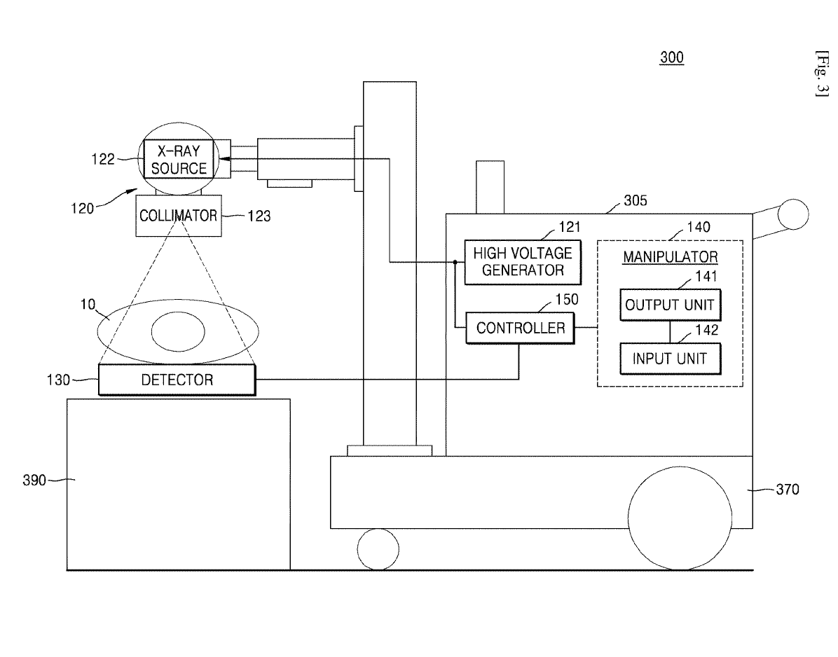

FIG. 4 is a block diagram illustrating a structure of the CT system 400. The detector 400 may be an embodiment of the detector 130 of FIGS. 1-3. The detector 400 may be an indirect type detector.

Referring to FIG. 4, the detector 400 may include a scintillator (not shown), a photodetecting substrate 410, a bias driver 430, a gate driver 450, and a signal processor 470.

The scintillator receives the X-ray radiated from the X-ray source 122 and converts the X-ray into light.

The photodetecting substrate 410 receives the light from the scitillator and converts the light into an electrical signal. The photodetecting substrate 410 may include gate lines GL, data lines DL, TFTs 412, photodiodes 414, and bias lines BL.

The gate lines GL may be formed in the first direction DR1, and the data lines DL may be formed in the second direction DR2 that crosses the first direction DR1. The first direction DR1 and the second direction DR2 may intersect perpendicularly to each other. FIG. 4 shows four gate lines GL and four data lines DL as an example.

The TFTs 412 may be arranged as a matrix in the first and second directions DR1 and DR2. Each of the TFTs 412 may be electrically connected to one of the gate lines GL and one of the data lines DL. A gate of the TFT 412 may be electrically connected to the gate line GL, and a source of the TFT 412 may be electrically connected to the data line DL. In FIG. 4, sixteen TFTs 412 (in a 4×4 arrangement) are shown as an example.

The photodiodes 414 may be arranged as a matrix in the first and second directions DR1 and DR2 so as to respectively correspond to the TFTs 412. Each of the photodiodes 414 may be electrically connected to one of the TFTs 412. An N-side electrode of each of the photodiodes 414 may be electrically connected to a drain of the TFT 412. FIG. 4 shows sixteen photodiodes 414 (in a 4×4 arrangement) as an example.

The bias lines BL are electrically connected to the photodiodes 414. Each of the bias lines BL may be electrically connected to P-side electrodes of an array of photodiodes 414. For example, the bias lines BL may be formed to be substantially parallel with the second direction DR2 so as to be electrically connected to the photodiodes 414. On the other hand, the bias lines BL may be formed to be substantially parallel with the first direction DR1 so as to be electrically connected to the photodiodes 414. FIG. 4 shows four bias lines BL formed along the second direction DR2 as an example.

The bias driver 430 is electrically connected to the bias lines BL so as to apply a driving voltage to the bias lines BL. The bias driver 430 may selectively apply a reverse bias voltage or a forward bias voltage to the photodiodes 414. A reference voltage may be applied to the N-side electrodes of the photodiodes 414. The reference voltage may be applied via the signal processor 470. The bias driver 430 may apply a voltage that is less than the reference voltage to the P-side electrodes of the photodiodes 414 so as to apply a reverse bias voltage to the photodiodes 414. On the other hand, the bias driver 430 may apply a voltage that is greater than the reference voltage to the P-side electrodes of the photodiodes 414 so as to apply a forward bias voltage to the photodiodes 414.

The gate driver 450 is electrically connected to the gate lines GL and thus may apply gate signals to the gate lines GL. For example, when the gate signals are applied to the gate lines GL, the TFTs 412 may be turned on by the gate signals. On the other hand, when the gate signals are not applied to the gate lines GL, the TFTs 412 may be turned off.

The signal processor 470 is electrically connected to the data lines DL. When the light received by the photodetecting substrate 410 is converted into the electrical signal, the electrical signal may be read out by the signal processor 470 via the data lines DL.

An operation of the detector 400 will now be described. During the operation of the detector 400, the bias driver 430 may apply the reverse bias voltage to the photodiodes 414.

While the TFTs 412 are turned off, each of the photodiodes 414 may receive the light from the scintillator and generate electron-hole pairs to accumulate electric charges. The amount of electric charge accumulated in each of the photodiodes 414 may correspond to the intensity of the received X-ray.

Then, the gate driver 450 may sequentially apply the gate signals to the gate lines GL along the second direction DR2. When a gate signal is applied to a gate line GL and thus TFTs 412 connected to the gate line GL are turned on, photocurrents may flow into the signal processor 470 via the data lines DL due to the electric charges accumulated in the photodiodes 414 connected to the turned-on TFTs 412.

The signal processor 470 may convert the received photocurrents into image data. The signal processor 470 may output the image data to the outside. The image data may be in the form of an analog signal or a digital signal corresponding to the photocurrents.

Although not shown in FIG. 4, if the detector 400 shown in FIG. 4 is a wireless detector, the detector 400 may further include a battery unit and a wireless communication interface unit.

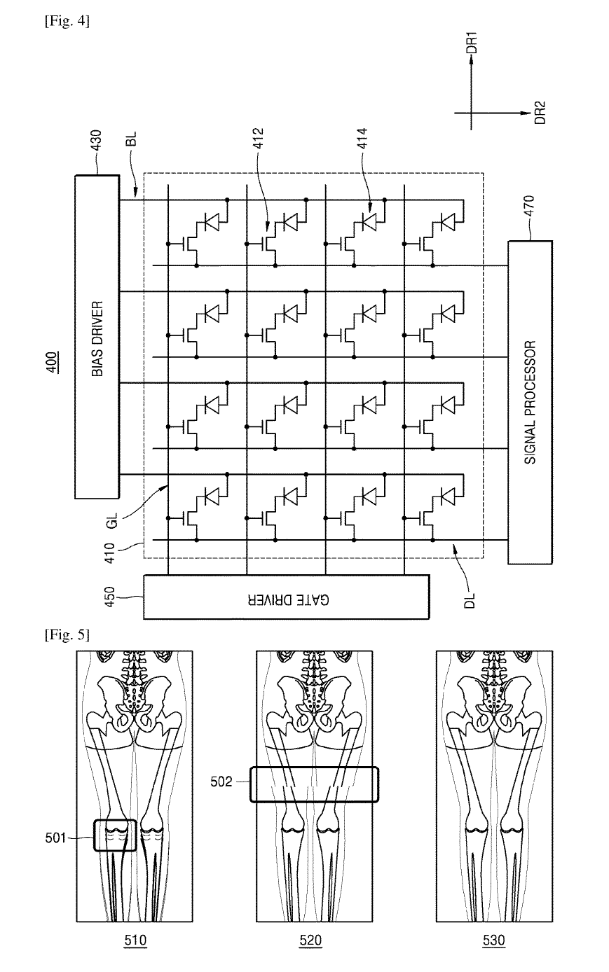

FIG. 5 explains a result of synthesizing a plurality of images having an overlapped region, according to an embodiment.

When an X-ray penetrates through an object, the X-ray is attenuated according to the property and distance of the object. Due to the attenuating characteristics of X-rays, X-ray imaging apparatuses capable of examining an internal shape of a human body or an object are widely used for non-destructive examination for a medical use or an industrial use.

A captured region of an object that may be captured at a time by an X-ray imaging apparatus may be limited to a certain region on the object depending on the capturing accuracy or object resolution of the apparatus. Thus, the X-ray imaging apparatus may acquire an image having a wider area or a higher resolution by combining a plurality of captured images according to an image stitching technique.

For example, the X-ray imaging apparatus may acquire a plurality of images by photographing the lower part of a human body, and combine the plurality of images together to generate a synthesis image. In this case, the synthesis image may include a mismatched region due to the combination of the plurality of images.

An image 510 of FIG. 5 is a synthesis image having a mismatched region 501 due to a location error of the X-ray imaging apparatus. An image 520 of FIG. 5 is a synthesis image having a mismatched region 502 due to a magnification ratio error for at least one of the plurality of images.

Thus, the X-ray imaging apparatus needs to generate a seamless image in which images accurately overlap each other, as shown in an image 530 of FIG. 5.

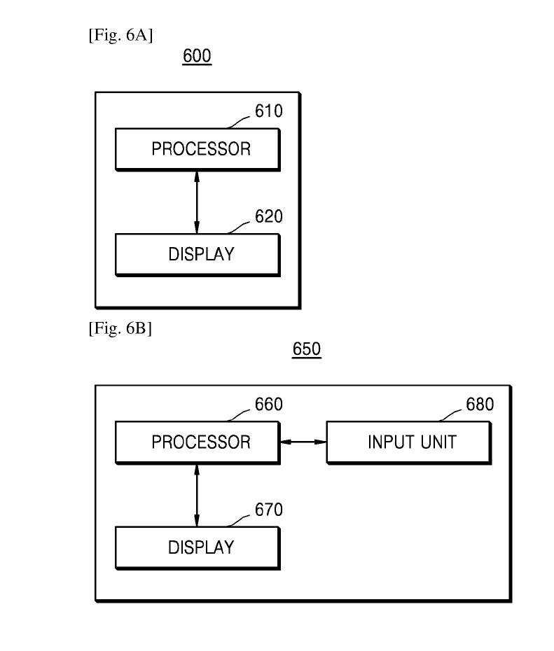

FIG. 6A is a block diagram of a structure of a medical image processing apparatus 600 according to an embodiment.

According to an embodiment, the medical image processing apparatus 600 may include a processor 610 and a display 620. It will be understood by one of ordinary skill in the art to which this embodiment pertains that the medical image processing apparatus 600 may include other general-use components in addition to the components illustrated in FIG. 6A.

The medical image processing apparatus 600 of FIG. 6A may be the X-ray apparatus 100 of FIG. 1, the workstation 110 of FIG. 1, the medical apparatus 164 of FIG. 1, the portable terminal 166 of FIG. 1, a medical imaging apparatus, a medical server, or any computing device capable of using and processing a medical image. In detail, the processor 610 of the medical image processing apparatus 600 of FIG. 6A may correspond to the controller 150 of the X-ray apparatus 100 of FIG. 1 or the controller 113 of the workstation 110 of FIG. 1. The display 620 of the medical image processing apparatus 600 of FIG. 6A may correspond to the output unit 141 of the X-ray apparatus 100 of FIG. 1 or the output unit 111 of the workstation 110 of FIG. 1. A description of FIG. 6A that is the same as given above with reference to FIG. 1 will not be repeated herein. The structure of the medical image processing apparatus 600 of FIG. 6A will now be described.

The processor 610 may acquire a first image and a second image captured by radiating an X ray to an object. The first image and the second image are captured by consecutively photographing a predetermined part of the object. The processor 610 may generate a synthesis image by overlapping a first region of the first image with a second region of the second image. In detail, the processor 610 may generate a synthesis image by overlapping a first region of the first image corresponding to a predetermined region of the object with a second region of the second image corresponding to the predetermined region of the object. The terms “first image” and “second image” described with reference to FIGS. 6A-10B may denote captured images commonly including a predetermined region of an object.

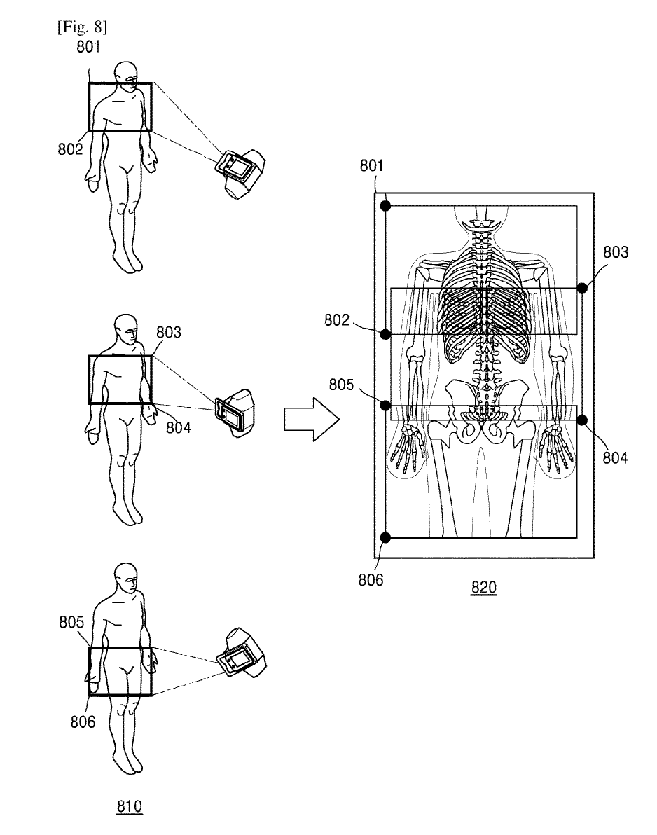

The processor 610 may generate a synthesis image by combining a plurality of images by using location information of a detector located when capturing each of the plurality of images. The detector is an apparatus for detecting an X-ray transmitted by the object. In detail, the processor 610 may receive first location information representing a location of the detector during capturing of the first image, and receive second location information representing a location of the detector during capturing of the second image. For example, the first location information may be first height coordinate information of the detector, and the second location information may be second height coordinate information of the detector. The processor 610 may determine overlapping regions of the first image and the second image, based on the first location information and the second location information. The processor 610 may determine the first region of the first image and the second region of the second image as the overlapping regions and overlap the overlapping regions to generate a synthesis image. This will be described in detail with reference to FIG. 8.

The processor 610 may acquire information of matching accuracy representing the degree to which the overlapping regions corresponding to the first region of the first image and the second region of the second image match with each other. The information of the matching accuracy may include, but is not limited to, at least one of information about a length of an overlap between the first image and the second image on the synthesis image, information about a location of an overlapped region on the synthesis image, and information about the matching accuracy representing a match rate between the first region of the first image and the second region of the second image. The matching accuracy representing the match rate between the first region of the first image and the second region of the second image may be expressed in percentage.

The display 620 may output an acquired image. The display 620 may display not only an image but also various pieces of information that are processed by the medical image processing apparatus 600, on a screen via a graphical user interface (GUI). The medical image processing apparatus 600 may include two or more displays 620 according to embodiments.

The first image and the second image may be acquired by the medical image processing apparatus 600 directly photographing the object, or may be acquired by an external apparatus physically independent from the medical image processing apparatus 600.

The external apparatus acquires, stores, processes, or utilizes an image and data related with the image. Thus, the external apparatus may be a medical imaging apparatus, a medical server, a portable terminal, or any computing device capable of utilizing and processing medical images. For example, the external apparatus may be a medical diagnosis apparatus included in a medical institution such as a hospital. The external apparatus may be, for example, a server included in a hospital for recoding and storing medical treatment histories of patients, or a medical imaging apparatus used by medical doctors in a hospital to read medical images.

The display 620 may display, on the synthesis image, a marker indicating a location of an overlapped region between the first image and the second image. Alternatively, the display 620 may display the marker indicating the location of the overlapped region between the first image and the second image, together with the synthesis image. The display 620 may also display at least one of information about a length of the overlapped region between the first image and the second image on the synthesis image and information about a location of the overlapped region on the synthesis image.

The display 620 may display information representing whether matching between the first image and the second image succeeds, based on the matching accuracy. In detail, when the first region of the first image matches with the second region of the second image, the display 620 may display information representing that the matching has succeeded. The information representing that the matching has succeeded may include, but is not limited to, a text representing a match rate of 100%. On the other hand, when the first region of the first image mismatches with the second region of the second image, the display 620 may display information representing that the matching has failed. The information representing that the matching has failed may include a text representing a match rate or a mismatch rate.

The processor 610 may distinguishably set at least one of a color, shape, and pattern of the marker representing the location of the overlapped region between the first image and the second image, based on the matching accuracy. The display 620 may distinguish the case where the first region of the first image matches with the second region of the second image from the case where the first region of the first image mismatches with the second region of the second image, and may display matching accuracy information. In detail, when the first region of the first image mismatches with the second region of the second image, the display 620 may distinguishably display a predetermined portion corresponding to a mismatch portion between the first region of the first image and the second region of the second image. For example, the predetermined portion corresponding to the mismatch portion may be displayed in a different color from an existing color of the synthesis image. Alternatively, when the first region of the first image mismatches with the second region of the second image, the display 620 may change and display a color or shape of the marker indicating the location of the overlapped region between the first and second images.

The medical image processing apparatus 600 may capture a plurality of images and combine respective overlapping regions of the plurality of images with each another to generate a synthesis image. In this case, the medical image processing apparatus 600 may provide matching accuracy information of the overlapping regions of the plurality of images and thus may provide more accurate information to a patient.

The medical image processing apparatus 600 may further include a central processor to control overall operations of the processor 610 and the display 620. The central processor may be implemented by an array of a plurality of logic gates, or by a combination of a general-use microprocessor and a memory in which a program to be executed by the general-use microprocessor is stored. It will also be understood by one of ordinary skill in the art to which this embodiment pertains that the central processor may be implemented by other types of hardware.

FIG. 6B is a block diagram of a structure of a medical image processing apparatus 650 according to another embodiment.

The medical image processing apparatus 650 may include a processor 660, a display 670, and an input unit 680. It will be understood by one of ordinary skill in the art to which this embodiment pertains that the medical image processing apparatus 650 may include other general-use components in addition to the components illustrated in FIG. 6B.

The medical image processing apparatus 650 of FIG. 6B may further include the input unit 680, compared with the medical image processing apparatus 600 of FIG. 6A. The processor 660 and the display 670 of the medical image processing apparatus 650 of FIG. 6B respectively correspond to the processor 610 and the display 620 of the medical image processing apparatus 600 of FIG. 6A.

The input unit 680 of the medical image processing apparatus 650 of FIG. 6B may correspond to the input unit 142 of the X-ray apparatus 100 of FIG. 1 or the input unit 112 of the workstation 110 of FIG. 1. A description of FIG. 6B that is the same as given above with reference to FIGS. 1 and 6A will not be repeated herein. The structure of the medical image processing apparatus 650 of FIG. 6B will now be described.

The input unit 680 refers to a device via which a user inputs data for controlling the medical image processing apparatus 650. The input unit 680 may include, but is not limited to, hardware structures such as a keypad, a mouse, a touch panel, a touch screen, a track ball, and a jog switch. The input unit 680 may further include various input units, such as a voice recognition sensor, a gesture recognition sensor, a fingerprint recognition sensor, an iris recognition sensor, a depth sensor, and a distance sensor.

The input unit 680 may receive a user input for correcting the range of at least one of the first region of the first image and the second region of the second image. The processor 660 may correct at least one image, based on the user input.

The input unit 680 may generate a UI screen image for receiving a command or data from a user, and the display 670 may display the UI screen image. For example, the input unit 680 may receive at least one of an input of correcting a section of an overlapped region between the first image and the second image and an input of adjusting a magnification ratio of the first image or the second image. The UI screen image may include a magnified image of the overlapped region between the first image and the second image and an icon for manipulating the first image or the second image.

In more detail, the input unit 680 may receive a manipulation signal generated due to a user touch input via various input tools. The input unit 680 may receive an input of correcting the section of the overlapped region between the first image and the second image displayed on the screen, via a hand or a physical tool of a user, and the processor 660 may correct the section of the overlapped region according to the user input.

The medical image processing apparatus 650 may further include a memory (not shown) and a communicator (not shown). The memory (not shown) may store, for example, an image and data related with the image (for example, an X-ray image and diagnosis data of a patient regarding the X-ray image) and data transmitted from an external apparatus to the medical image processing apparatus 650. The data transmitted by the external apparatus to the medical image processing apparatus 650 may include patient-related information, data necessary for diagnosing and treating patients, histories of previous treatments of patients, and a medical worklist (MWL) corresponding to diagnosis instructions for patients, and the like. The memory (not shown) may store information of the matching accuracy representing the degree to which the overlapping regions of the first image and the second image match with each other. The memory may also store a synthesis image on which the information of the matching accuracy is displayed. The memory may store a synthesis image in which the overlapping regions of the first image and the second image have been corrected based on a user input.

The communicator may receive data from the external apparatus and/or transmit data to the external apparatus. For example, the communicator may be connected to the X-ray apparatus 100, the workstation 110, the server 162, the medical apparatus 164, and the portable terminal 166 via a Wi-Fi or Wi-Fi Direct (WFD) communication network.

The medical image processing apparatus 650 provides the matching accuracy information of the overlapping regions of the plurality of images and provides a UI screen image via which a user is able to correct predetermined mismatched portions on the overlapping regions. The medical image processing apparatus 650 may provide a more accurate image to the user by correcting the synthesis image according to a user input of correcting the mismatched portions.

The medical image processing apparatus 650 may further include a central processor to control overall operations of the processor 660, the display 670, and the input unit 680. The central processor may be implemented by an array of a plurality of logic gates, or by a combination of a general-use microprocessor and a memory in which a program to be executed by the general-use microprocessor is stored. It will also be understood by one of ordinary skill in the art to which this embodiment pertains that the central processor may be implemented by other types of hardware.

Various operations or applications that the medical image processing apparatuses 600 and 650 execute will now be described. However, matters to be clearly understood and expected by one of ordinary skill in the art to which the present invention pertains may be understood by typical implementations even when none of the processors 610 and 660, the displays 620 and 670, and the input unit 680 is specified, and the scope of the present invention is not limited by the titles or physical/logical structures of specified components.

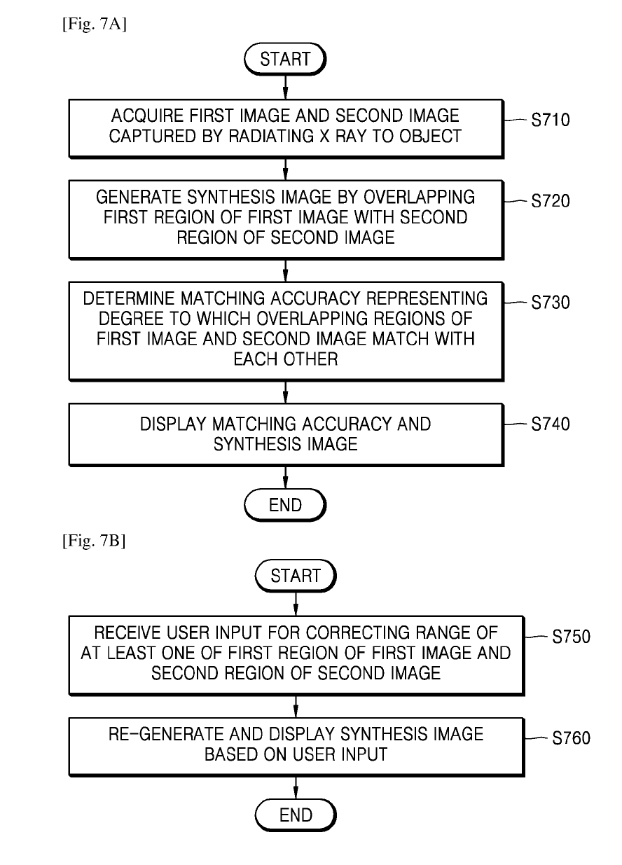

FIG. 7A is a flowchart of a medical image processing method according to an embodiment.

An operation of the medical image processing apparatus 600 will now be described, but this description is not be limited to the medical image processing apparatus 600 but may be equally applied to the medical image processing apparatus 650.

In operation S710 of FIG. 7A, the medical image processing apparatus 600 may acquire a first image and a second image. The first image and the second image are captured by radiating an X ray to an object, and are images captured by consecutively photographing a predetermined part of the object.

In operation S720, the medical image processing apparatus 600 may generate a synthesis image by overlapping a first region of the first image with a second region of the second image.

The medical image processing apparatus 600 may receive first location information representing a location of a detector for detecting an X-ray transmitted by the object during capturing of the first image, and receive second location information representing a location of the detector during capturing of the second image. The medical image processing apparatus 600 may determine the first region of the first image and the second region of the second image based on the first location information and the second location information, and overlap the first region with the second region to generate the synthesis image.

In operation S730, the medical image processing apparatus 600 may determine matching accuracy representing the degree to which the overlapping regions of the first image and the second image match with each other. The medical image processing apparatus 600 may determine at least one of information about a length of an overlap between the first image and the second image on the synthesis image, information about a location of an overlapped region on the synthesis image, and the matching accuracy representing a match rate between the first region of the first image and the second region of the second image, but the inventive concept is not limited thereto.

In operation S740, the medical image processing apparatus 600 may display the matching accuracy and the synthesis image. The medical image processing apparatus 600 may display a marker indicating a location of an overlapped region between the first image and the second image, together with the synthesis image.

The medical image processing apparatus 600 may display at least one of the information about the length of the overlap between the first image and the second image on the synthesis image and the information about the location of the overlapped region on the synthesis image.

The medical image processing apparatus 600 may display information representing whether matching between the first image and the second image has succeeded, based on the matching accuracy.

When the first region of the first image mismatches with the second region of the second image, the medical image processing apparatus 600 may change and display a color or shape of the marker indicating the location of the overlapped region between the first and second images. The medical image processing apparatus 600 may distinguishably set and display at least one of a color, shape, and pattern of the marker representing the location of the overlapped region between the first image and the second image, based on the matching accuracy.

FIG. 7B is a flowchart of a medical image processing method according to another embodiment.

In operation S750 of FIG. 7B, the medical image processing apparatus 650 may receive a user input for correcting the range of at least one of the first region of the first image and the second region of the second image. The medical image processing apparatus 650 may receive an input of correcting a section of the overlapped region between the first image and the second image. The medical image processing apparatus 650 may receive an input of adjusting a magnification ratio of the first image or the second image.

In operation S760, the medical image processing apparatus 650 may correct at least one image based on the user input, and re-generate a synthesis image by using a result of the correction. The medical image processing apparatus 650 may display the re-generated synthesis image.

There may be provided a computer-readable recording medium having recorded thereon a program for the method of executing operations S710-S740 described above with reference to FIG. 7A. There may also be provided a computer-readable recording medium having recorded thereon a program for the method of executing operations S710-S760 described above with reference to FIG. 7B.

FIG. 8 explains a method of synthesizing a plurality of images captured according to locations on an object, according to an embodiment.

Referring to FIG. 8, the medical image processing apparatus 650 may acquire the plurality of images by photographing the object by using an X-ray. The medical image processing apparatus 650 may repeatedly photograph a predetermined region on the object in order to acquire consecutive images of the object. The medical image processing apparatus 650 may acquire an image into which the plurality of images are combined. The medical image processing apparatus 650 may include a source that radiates an X-ray to the object, a detector that detects an X-ray transmitted by the object, a processor that controls operations of the source and the detector and overall operations of the medical image processing apparatus 650, and a display that displays an image.

As shown in a image 810 of FIG. 8, the source may radiate an X-ray to a head part, a torso part, and a leg part of a human, and the detector may detect X-rays respectively transmitted by the head part, the torso part, and the leg part of the human. The processor may acquire a first image, a second image, and a third image, based on the detected X-rays.

The processor may receive, from the detector, first location information representing a location of the detector during photography of the head part, second location information representing a location of the detector during photography of the torso part, and third location information representing a location of the detector during photography of the leg part. Location information of the detector may be, but is not limited to, height information or coordinate information of a location of the detector. For example, the processor may acquire the first location information of the detector as first height coordinate information 801 and 802 of the detector, the second location information of the detector as second height coordinate information 803 and 804 of the detector, and the third location information of the detector as third height coordinate information 805 and 806 of the detector.

The processor may detect an overlapped region between the first image and the second image by using the first height coordinate information 801 and 802 and the second height coordinate information 803 and 804. The processor may also detect an overlapped region between the second image and the third image by using the second height coordinate information 803 and 804.

As shown in a image 820 of FIG. 8, the processor may generate a synthesis image by combining the first image with the second image by using the overlapped region between the first image and the second image and combining the second image with the third image by using the overlapped region between the second image and the third image.

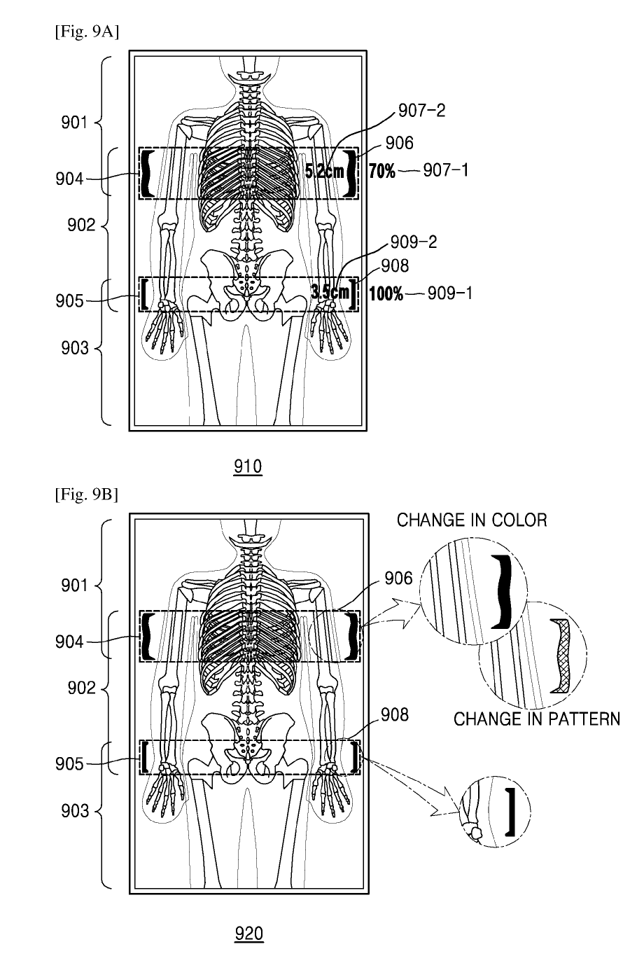

FIG. 9A explains a screen image that is output to a medical image processing apparatus and displays matching accuracy, according to an embodiment.

The medical image processing apparatus 650 may acquire a first image 901 corresponding an upper part of an object, a second image 902 corresponding a torso part of the object, and a third image 903 corresponding a lower part of the object. As shown in a synthesis image 910 of FIG. 9A, an overlapped region 904 exists between the first image 901 and the second image 902, and an overlapped region 905 exists between the second image 902 and the third image 903. The medical image processing apparatus 650 may generate the synthesis image 910 by overlapping respective overlapping regions of images. The medical image processing apparatus 650 may display the synthesis image 910 and matching accuracy.

Information of the matching accuracy may include at least one of information about a length of the overlapped region 904 between the first image 901 and the second image 902, information about a length of the overlapped region 905 between the second image 902 and the third image 903, information about a location of the overlapped region 904 on the synthesis image 910, information about locations of the second image 902 and the third image 903 on the synthesis image 910, information about a matching accuracy probability of the overlapped region 904 between the first image 901 and the second image 902, and information about a matching accuracy probability of the overlapped region 905 between the second image 902 and the third image 903. The matching accuracy representing a match rate of the respective overlapping regions of the plurality of images may be expressed in percentage. It will be understood by one of ordinary skill in the art to which this embodiment pertains that the information of the matching accuracy may include other matching accuracy information of the synthesis image 910 in addition to the aforementioned information.

As shown in the synthesis image 910 of FIG. 9A, the medical image processing apparatus 650 may display a marker 906 indicating the location of the overlapped region 904 between the first image 901 and the second image 902, information 907-1 representing that the matching accuracy between the first image 901 and the second image 902 is 70%, and information 907-2 representing that the length of the overlapped region 904 is 5.2 cm.

The medical image processing apparatus 650 may also display a marker 908 indicating the location of the overlapped region 905 between the second image 902 and the third image 903, information 909-1 representing that the matching accuracy between the second image 902 and the third image 903 is 100%, and information 909-2 representing that the length of the overlapped region 905 is 3.5 cm.

FIG. 9B explains a screen image that is output to a medical image processing apparatus and displays matching accuracy, according to another embodiment.

A synthesis image 920 of FIG. 9B is the same as the synthesis image 910 of FIG. 9A. A description of FIG. 9B that is the same as given above with reference to FIG. 9A will not be repeated herein. Reference numerals shown in the synthesis image 910 of FIG. 9A are equally applied to the synthesis image 920 of FIG. 9B, and thus repeated descriptions thereof will be omitted.

When overlapping regions corresponding to a predetermined part of an object on a plurality of images match with each other, the medical image processing apparatus 650 may display information representing that matching between the plurality of images has succeeded. When the overlapped regions corresponding to the predetermined part of the object on the plurality of images mismatch with each other, the medical image processing apparatus 650 may display information representing that matching between the plurality of images has failed.

The medical image processing apparatus 650 may distinguish the case where a first region of a first image and a second region of a second image corresponding to the predetermined part of the object match with each other from the case where the first region of the first image and the second region of the second image mismatch with each other, and distinguishably set and display at least one of a color, shape, and pattern of a marker representing a location of an overlapped region between the first image and the second image.

Referring to the synthesis image 920 of FIG. 9B, when respective overlapped regions 904 of the first image 901 and the second image 902 do not match with each other, the medical image processing apparatus 650 may display the marker 906, representing the location of the overlapped regions 904, in red, or change and display the pattern of the marker 906. On other hand, when respective overlapped regions 905 of the second image 902 and the third image 903 match with each other, the medical image processing apparatus 650 may display the marker 908, representing the location of the overlapped regions 905, in blue. The above disclosure is only an example, and it will be understood by one of ordinary skill in the art to which this disclosure pertains that a marker may be displayed in various other manners according to a match or a mismatch between respective overlapped regions of a plurality of images.

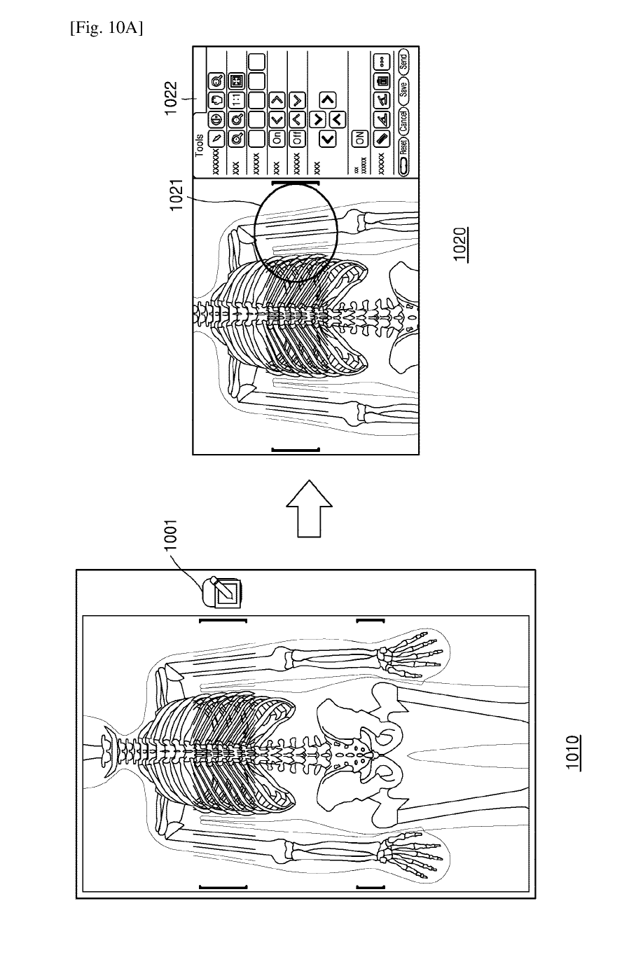

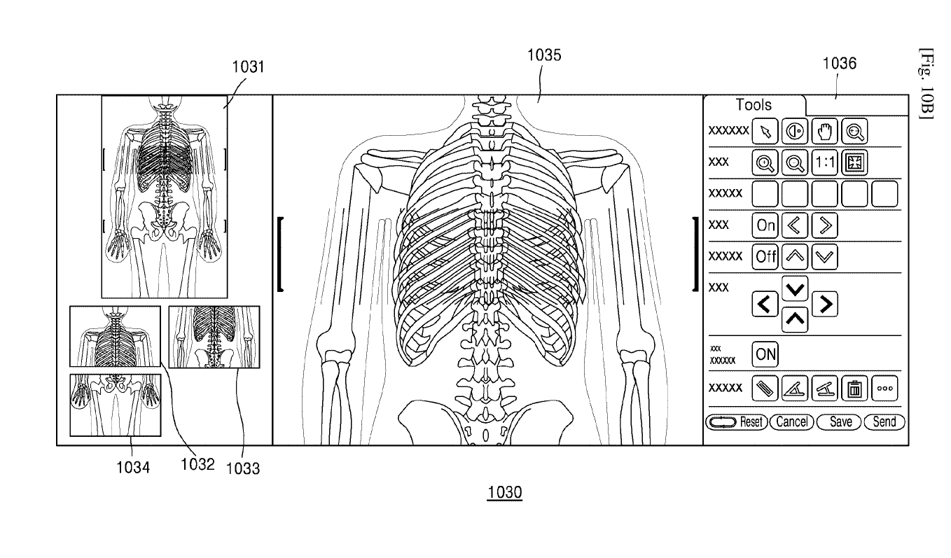

FIG. 10A explains a UI screen image via which a medical image processing apparatus corrects a synthesis image, according to an embodiment.

Referring to FIG. 10A, the medical image processing apparatus 650 may receive an input of selecting a button 1001, via a screen image 1010 on which the synthesis image is displayed. In response to the input of selecting the button 1001, the medical image processing apparatus 650 may change the screen image 1010, on which the synthesis image is displayed, to a UI screen image 1020 for correcting the synthesis image, and display the UI screen image 1020.

The UI screen image 1020 may include a magnified image of an overlapped region 1021 between the first image and the second image and icons 1022 for manipulating the first image or the second image. The medical image processing apparatus 650 may receive an input of manipulating the first image or the second image, from a user via the UI screen image 1020. For example, the medical image processing apparatus 650 may receive an input of correcting a section of an overlapped region between the first image and the second image, and may receive an input of adjusting a magnification ratio of the first image or the second image. The medical image processing apparatus 650 may correct the first or second image based on the user input, and re-generate a synthesis image by using a result of the correction.

FIG. 10B explains a UI screen image via which a medical image processing apparatus corrects a synthesis image, according to another embodiment.

The medical image processing apparatus 650 may change the screen image 1010 of FIG. 10A, on which the synthesis image is displayed, to a UI screen image 1030 for correcting the synthesis image, and display the UI screen image 1030.

A user may visually recognize some information from the UI screen image 1030 displayed by a display and may input a command or data via the UI screen image 1030. The UI screen image 1030 of FIG. 10B may include a synthesis image 1031, a first image 1032, a second image 1033, a third image 1034, and a magnified image 1035 of an overlapped region between the first image 1032 and the second image 1033. The UI screen image 1030 of FIG. 10B may include an icon 1036 used to manipulate the images displayed on the UI screen image 1030 or to correct the overlapped region between the first image 1032 and the second image 1033 on the magnified image 1035.

The UI screen image 1030 may include a touchpad coupled to a display panel included in the display. In this case, the UI screen image 1030 may be displayed on the display panel.

A UI may receive an input of correcting the range of an overlapped region between a first image and a second image or an input of adjusting magnification ratios of the first image and the second image, via a hand or a user or a physical tool (for example, a stylus pen) of the user. The medical image processing apparatus 650 may correct the first or second image based on the user input, and re-generate a synthesis image by using a result of the correction.

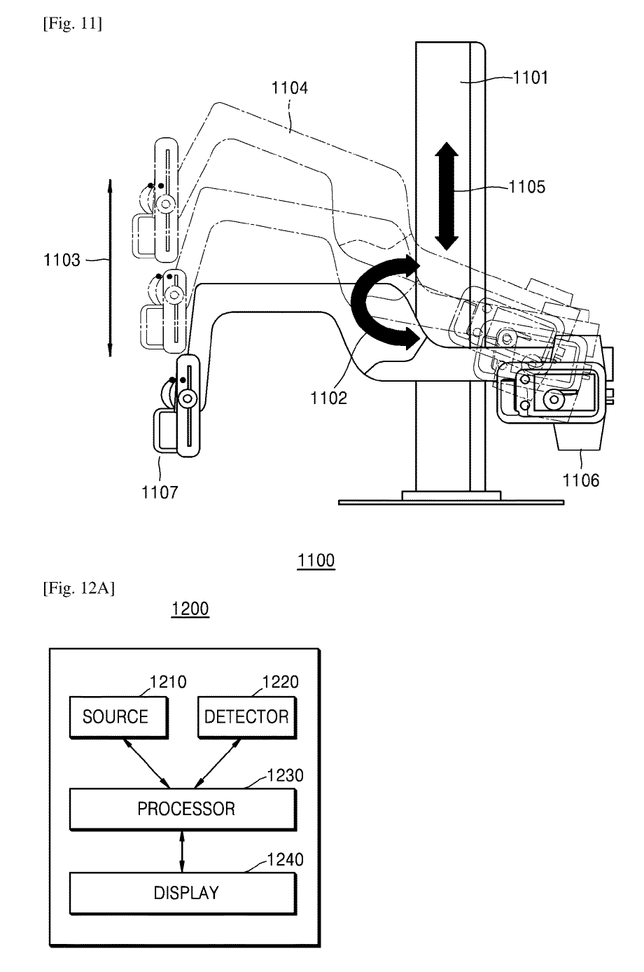

FIG. 11 explains an operation of an X-ray imaging apparatus according to an embodiment.

The X-ray imaging apparatus may include a device that generates an X-ray, and a device that detects an X-ray and converts the X-ray to an image. Examples of the X-ray imaging apparatus include a ceiling type X-ray imaging apparatus and a U-arm type X-ray imaging apparatus.

In the ceiling type X-ray imaging apparatus, an X-ray generating device is fixed to a ceiling. The ceiling type X-ray imaging apparatus operates widely and easily accesses a patient because of a high operational flexibility.

As shown in FIG. 11, a U-arm type X-ray imaging apparatus 1100 includes a source 1106 that generates an X ray and a detector 1107 that detects an X ray, and the source 1106 and the detector 1107 are fixed to an arm 1104 connected to an arm stand on the ground. The U-arm type X-ray imaging apparatus 1100 occupies a small space and is cheap in terms of equipment prices and installation costs, compared with ceiling type X-ray imaging apparatuses. However, since the source 1106 and the detector 1107 in the U-arm type X-ray imaging apparatus 1100 are connected together by the arm 1104, motions of the source 1106 and the detector 1107 are restricted.

Referring to FIG. 11, the arm 1104 included in the U-arm type X-ray imaging apparatus 1100 may rotate with respect to a support 1101 as indicated by an arrow 1102, and may move vertically as indicated by an arrow 1105. The detector 1107 located on an end of the arm 1104 may linearly move as indicated by an arrow 1103, in correspondence with the rotation or vertical motion of the arm 1104.

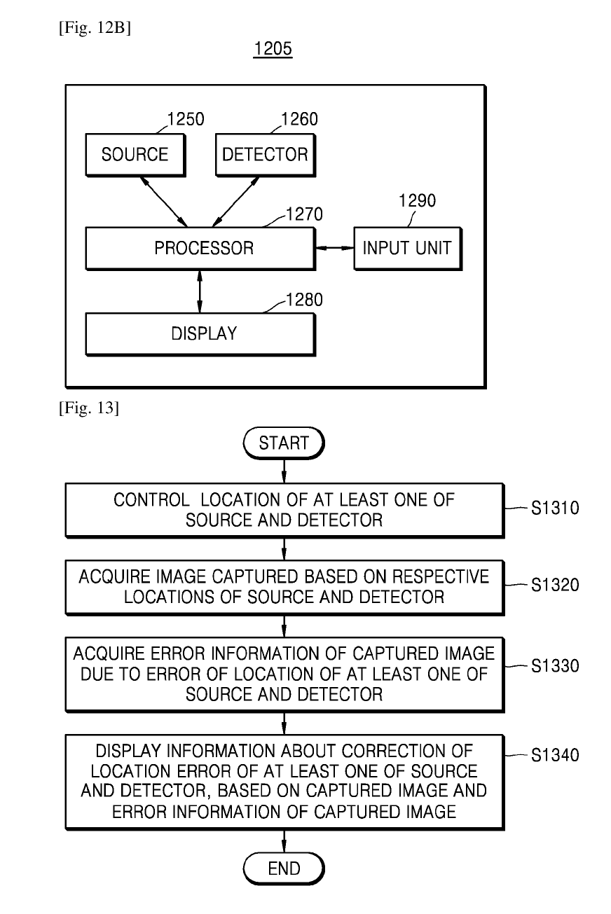

FIG. 12A is a block diagram of a structure of an X-ray imaging apparatus 1200 according to an embodiment.

The X-ray imaging apparatus 1200 may include a source 1210, a detector 1220, a processor 1230, and a display 1240. It will be understood by one of ordinary skill in the art to which this embodiment pertains that the X-ray imaging apparatus 1200 may include other general-use components in addition to the components illustrated in FIG. 12A.

The X-ray imaging apparatus 1200 of FIG. 12A may be the same as the X-ray apparatus 100 of FIG. 1. In detail, the source 1210, the detector 1220, the processor 1230, and the display 1240 of the X-ray imaging apparatus 1200 of FIG. 12A may respectively correspond to the X-ray radiator 120, the detector 130, the controller 150, and the output unit 141 of the X-ray apparatus 100 of FIG. 1. A description of FIG. 12A that is the same as given above with reference to FIG. 1 will not be repeated herein. A structure of the X-ray imaging apparatus 1200 of FIG. 12A will now be described.

The source 1210 may radiate an X-ray to an object, and the detector 1220 may detect an X-ray transmitted by the object.

The processor 1230 may control a location of at least one of the source 1210 and the detector 1220. For example, when the X-ray imaging apparatus 1200 is a U-arm type X-ray imaging apparatus 1200, the source 1210 and the detector 1220 may be physically coupled to an arm. The processor 1230 may control an angle of the arm in order to control the locations of the source 1210 and the detector 1220. The processor 1230 may also control an incidence angle of an X-ray radiated by the source 1210. The processor 1230 may acquire an image captured based on the location of the source 1210 and that of the detector 1220. The processor 1230 may acquire error information of an image due to at least one of location errors of the source 1210 and the detector 1220.

Before the processor 1230 controls the location of the source 1210 or the detector 1220, location information according to a location of each component of the X-ray imaging apparatus 1200 is necessary, and reference location information needs to be set. For example, the reference location information may include, but is not limited to, coordinate system information (x axis, y axis, and z axis) of a place on which the X-ray imaging apparatus 1200 is mounted, a height and a width of the place on which the X-ray imaging apparatus 1200 is mounted, an inclination of a bottom of the place on which the X-ray imaging apparatus 1200 is mounted, Source to Image Distance (SID) information, and Source to Object Distance (SOD) information. Setting of the reference location information is referred to as calibration. Since calibration is performed with an actually measured value, an error may exist in the actually measured value. Since the X-ray imaging apparatus 1200 sensitively reacts with even a change in external environments, an error may also be generated due to even external factors. An error of calibration may be caused by a location error of the X-ray imaging apparatus 1200.

The processor 1230 may generate a predicted image based on the location of the source 1210 and that of the detector 1220, compare the captured image with the predicted image, and acquire error information of the captured image according to a result of the comparison. The predicted image needs to be captured based on the location of the source 121, the location of the detector 1220, and a location of the object. In contrast with the predicted image, the captured image is an actually captured image in which a location error of the source 121, a location error of the detector 1220, and a location error of the object have been reflected.

The processor 1230 may correct at least one of the location errors of the source 1210 and the detector 1220, based on error information of a first image and error information of a second image.

The display 1240 may display information about correction of at least one location error, based on an image and error information of the image. For example, the display 1240 may display a UI screen image including information used to correct at least one of a source and a detector.

The processor 1230 may acquire a first image and a second image captured based on correction of location error of the at least one of the source and the detector. The processor 1230 may generate a synthesis image by overlapping a first region of the first image and a second region of the second image corresponding to a predetermined region of the object. The display 1240 may display the generated synthesis image.

Te X-ray imaging apparatus 1200 may include a central processor to control overall operations of the source 1210, the detector 1220, the processor 1230, and the display 1240. The central processor may be implemented by an array of a plurality of logic gates, or by a combination of a general-use microprocessor and a memory in which a program to be executed by the general-use microprocessor is stored.

FIG. 12B is a block diagram of a structure of an X-ray imaging apparatus 1205 according to another embodiment.

The X-ray imaging apparatus 1205 may include a source 1250, a detector 1260, a processor 1270, a display 1280, and an input unit 1290. It will be understood by one of ordinary skill in the art to which this embodiment pertains that the X-ray imaging apparatus 1205 may include other general-use components in addition to the components illustrated in FIG. 12B.

The X-ray imaging apparatus 1205 of FIG. 12B may further include the input unit 1290, compared with the X-ray imaging apparatus 1200 of FIG. 12A. The source 1250, the detector 1260, the processor 1270, and the display 1280 of the X-ray imaging apparatus 1205 of FIG. 12B respectively correspond to the source 1210, the detector 1220, the processor 1230, and the display 1240 of the X-ray imaging apparatus 1200 of FIG. 12A.

The input unit 1290 of the X-ray imaging apparatus 1205 of FIG. 12B may correspond to the input unit 142 of the X-ray apparatus 100 of FIG. 1 or the input unit 112 of the workstation 110 of FIG. 1. A description of FIG. 12B that is the same as given above with reference to FIGS. 1 and 12A will not be repeated herein. The structure of the X-ray imaging apparatus 1205 of FIG. 12B will now be described.

The input unit 1290 refers to a device via which a user inputs data for controlling the X-ray imaging apparatus 1205. The input unit 1290 may include, but is not limited to, hardware structures such as a keypad, a mouse, a touch panel, a touch screen, a track ball, and a jog switch.

The input unit 1290 may receive a user input of correcting at least one of location errors of the source 1250 and the detector 1260. The processor 1270 may correct the at least one location error by changing at least one of the locations of the source 1250 and the detector 1260, based on the user input.

The processor 1270 may acquire a first image captured based on a first location of the source 1250 and a first location of the detector 1260. The processor 1270 may acquire error information of the first image due to at least one of respective location errors of the source 1250 and the detector 1260. The processor 1270 may determine a magnification ratio of the first image, which is captured based on the first location of the source 1250 and the first location of the detector 1260. For example, the processor 1270 may determine the magnification ratio of the first image by using a distance between the source 1250 and the detector 1260 and a distance between the source 1250 and an object. The magnification ratio of the first image may be calculated using Equation 1 below. The distance between the source 1250 and the detector 1260 may be calculated from the respective locations of the source 1250 and the detector 1260. The distance between the source 1250 and the object may be calculated from the respective locations of the source 1250 and the object.

### Math FIG. 1

$$Magnification ratio = \frac{distance between source and detector}{distance between source and object}$$

It will be understood by one of ordinary skill in the art to which this embodiment pertains that the magnification ratio of the first image may be calculated using other variables than Math 1.

The processor 1270 may generate a first predicted image by predicting an image that is to be captured based on the first location of the source 1250, the second location of the detector 1260, and the location of the object.

The processor 1270 may compare the first image with the first predicted image and acquire error information of the first image.

The input unit 1290 may generate a UI screen image for receiving a command or data from a user, and the display 1280 may display the UI screen image. The display 1280 may display a UI screen image for correcting at least one of the location errors of the source 1250 and the detector 1260. An acquired image and error information of the acquired image may be displayed on the UI screen image.

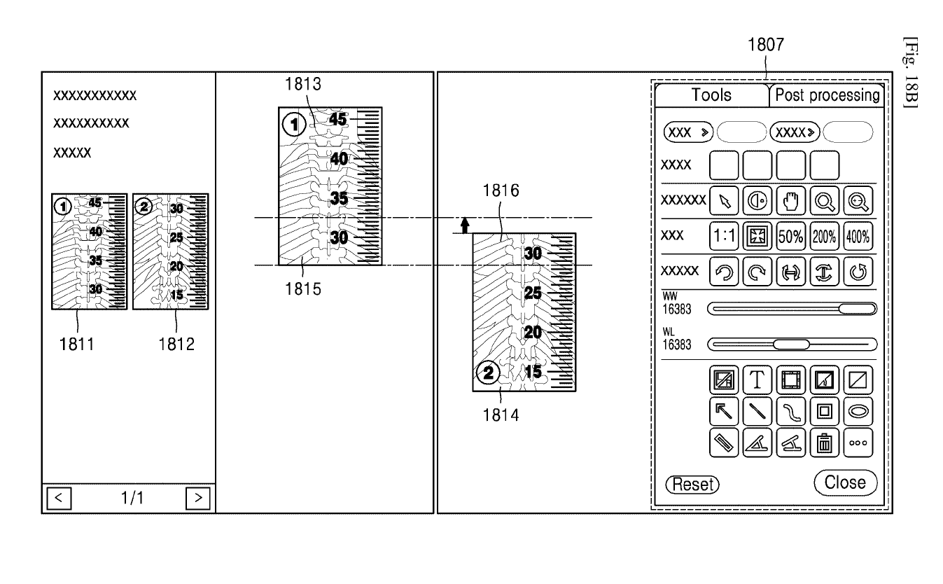

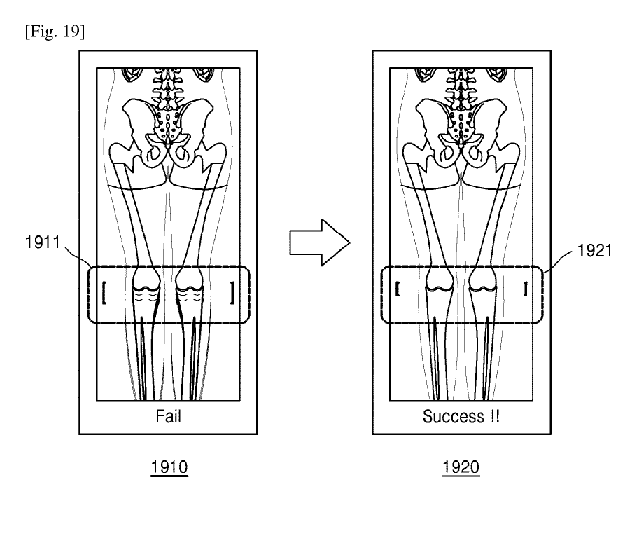

For example, the input unit 1290 may receive a user input of moving the first image or the second image based on an overlapped region between the first image and the second image. In detail, when an overlapped region between the first image and the second image exceeds a reference overlapped region, the input unit 1290 may receive a user input of moving the first image (or the second image) so that the overlapped region matches with the reference overlapped region. This will be described later in detail with reference to FIGS. 18A-19.

As another example, the input unit 1290 may receive a user input for correcting an error due to a difference between the magnification ratios of a captured image and a predicted image. The processor 1270 may detect the error due to the difference between the magnification ratios of the captured image and the predicted image. The processor 1270 may correct the magnification ratio of the captured image to the magnification ratio of the predicted image, based on the user input.

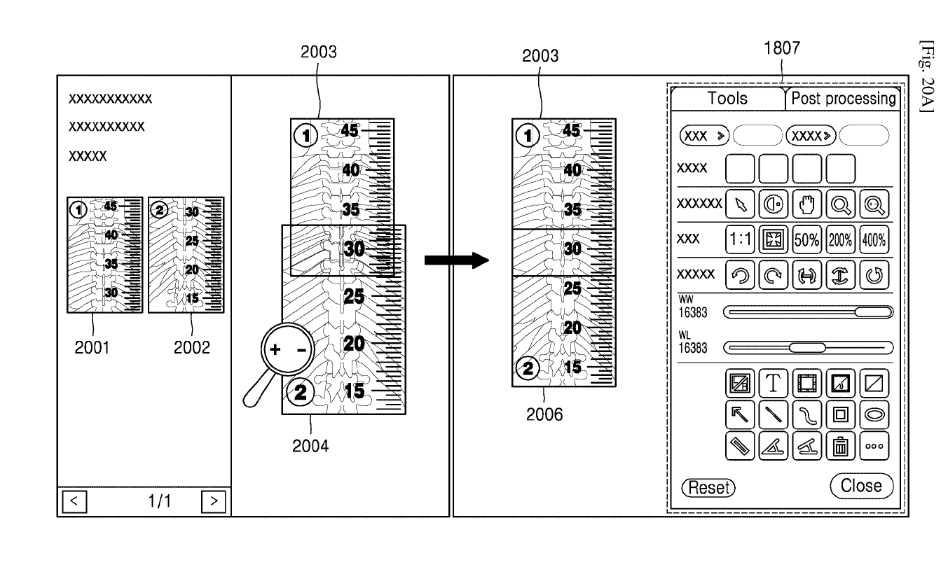

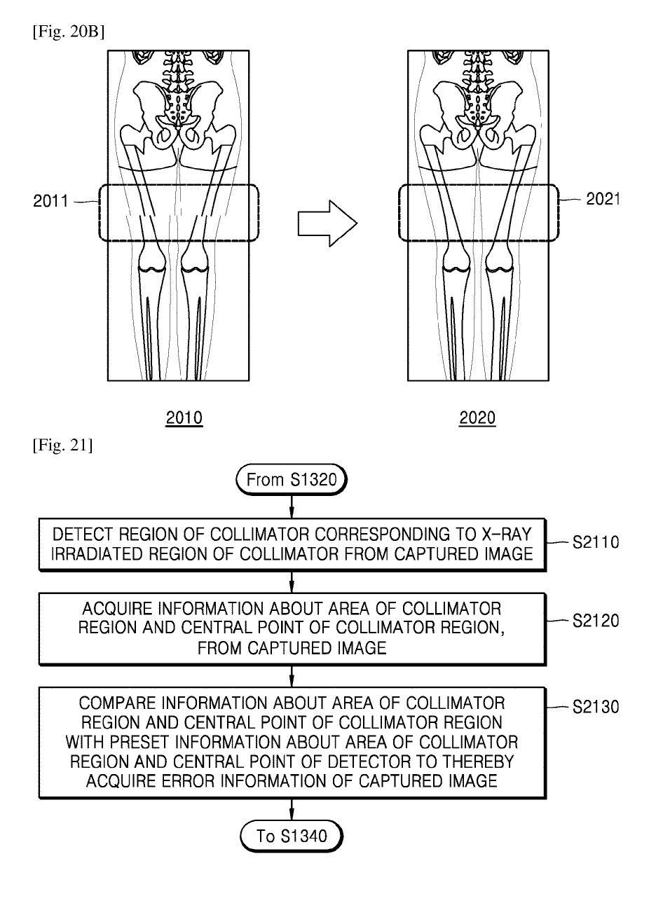

In detail, the input unit 1290 may receive a user input of correcting the magnification ratio of the first image to a magnification ratio of a predicted image for the first image and correcting the magnification ratio of the second image to a magnification ratio of a predicted image for the second image. In more detail, when the magnification ratio of the first image is greater than the magnification ratio of the predicted image for the first image, the input unit 1290 may receive a user input of reducing the magnification ratio of the first image. This will be described later in detail with reference to FIGS. 20A and 20B.

As another example, the processor 1270 may detect a region of a collimator corresponding to an X-ray irradiated region of the collimator from the acquired image. The collimator adjusts a region irradiated with an X-ray. The processor 1270 may acquire preset information. The preset information may include information about the area of a preset region of the collimator and the central point of the detector 1260. The processor 1270 may acquire the information about the area of the preset region of the collimator and the central point of the detector 1260 from the captured image, and compare information about the area of the collimator region and the central point of the collimator region with the preset information to thereby acquire error information of the captured image. In other words, the processor 1270 may compare the area of the collimator region with the area of the preset collimator region and the central point of the collimator region with the central point of the detector 1260 to thereby acquire the error information of the captured image. The display unit 1280 may display error information about the collimator region.

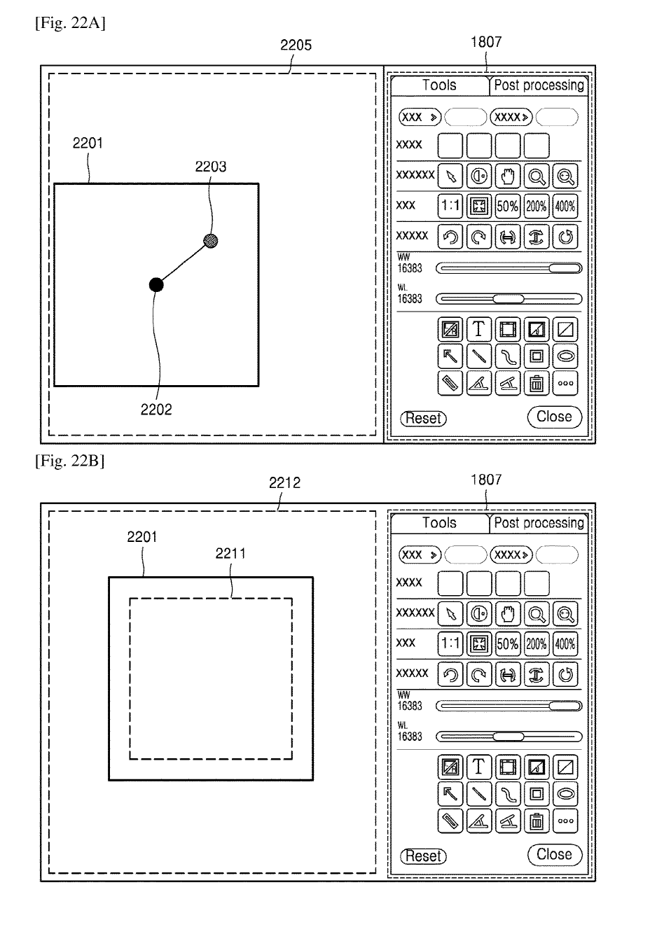

The input unit 1290 may receive at least one of a user input of adjusting the area of the collimator region and a user input of adjusting the central point of the collimator region. The processor 1270 may adjust at least one of the area of the collimator region and the central point of the collimator region according to the received user input. In detail, the processor 1270 may move the central point of the collimator region to the central point of the detector 1260 and adjust the area of the collimator region to be equal to the area of the preset collimator region, based on the user input. This will be described later in detail with reference to FIGS. 21-22B.

As another example, the processor 1270 may detect a collimator region corresponding to the X-ray irradiated region of the collimator from the captured image. The processor 1270 may acquire first captured coordinate values representing coordinate values of a plurality of vertices of the collimator region from the captured image, and may calculate first predicted coordinate values representing predicted coordinate values of the plurality of vertices of the collimator region from a preset collimator region on the collimator, based on the locations of the source 1250 and the detector 1260. The processor 1270 may compare the first predicted coordinate values with the first captured coordinate values to determine the error information of the captured image. The display unit 1280 may display error information about the collimator region.

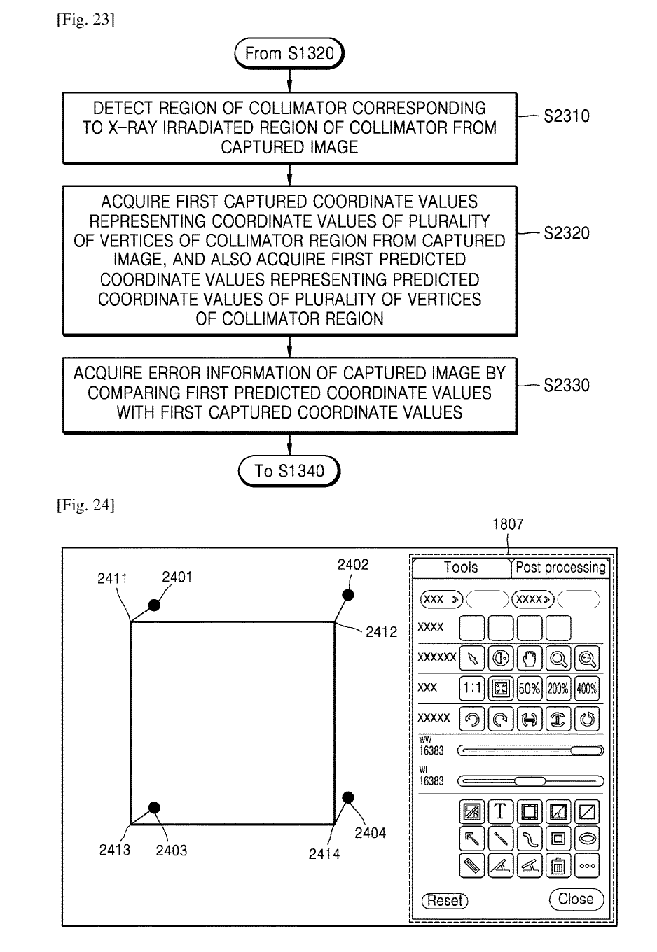

The input unit 1290 may receive a user input of moving the collimator region so that the first captured coordinate values are identical with the first predicted coordinate values, based on the error information. The processor 1270 may control the source 1250 to move the collimator region based on the user input. This will be described later in detail with reference to FIGS. 23 and 24.

The X-ray imaging apparatus 1205 may further include a memory (not shown) and a communicator (not shown). The memory (not shown) may store, for example, data related with a driving range and coordinate information of the X-ray imaging apparatus 1205 (for example, a driving range of the arm in the U-arm type X-ray imaging apparatus 1205 and coordinate information of a place on which the X-ray imaging apparatus 1205 is mounted) and data transmitted from an external apparatus to the X-ray imaging apparatus 1205. The memory may also store X-ray images related with the object.

The processor 1270 may control the object to be photographed based on the driving range of the X-ray imaging apparatus 1205 and location information of the place on which the X-ray imaging apparatus 1205 is mounted (for example, coordinate system information (x axis, y axis, and z axis) of the place on which the X-ray imaging apparatus 1205 is mounted, the height and width of the place on which the X-ray imaging apparatus 1205 is mounted, and the inclination of the bottom of the place on which the X-ray imaging apparatus 1205 is mounted, the SID information, and the SOD information), and component location information of the X-ray imaging apparatus 1205 (for example, the locations of the source 1250 and the detector 1260). The processor 1270 may correct the driving range and location information of the X-ray imaging apparatus 1205, based on the at least one of the location errors of the source 1250 and the detector 1260. For example, the processor 1270 may correct the coordinate system of the X-ray imaging apparatus 1205 by acquiring an error of the coordinate system of the X-ray imaging apparatus 1205 from the location errors of the source 1250 and the detector 1260. The memory may store the corrected driving range and the corrected location information. The processor 1270 may acquire images captured using the corrected driving range, the corrected location information, and at least one of the location errors of the source 1250 and the detector 1260 of a corrected X-ray imaging apparatus 1205. The processor 1270 may improve a matching rate of acquired images by combining the acquired images.

The communicator may receive data from the external apparatus and/or transmit data to the external apparatus. The communicator may transmit, for example, an image acquired according to location error correction of the X-ray imaging apparatus 1205 and location information of the X-ray imaging apparatus 1205 to an external apparatus or receive images related with the object from the external apparatus.

The X-ray imaging apparatus 1205 may include a central processor to control overall operations of the source 1250, the detector 1260, the processor 1270, the display 1280, and the input unit 1290. The central processor may be implemented by an array of a plurality of logic gates, or by a combination of a general-use microprocessor and a memory in which a program to be executed by the general-use microprocessor is stored. It will also be understood by one of ordinary skill in the art to which this embodiment pertains that the central processor may be implemented by other types of hardware.

Various operations or applications that an X-ray imaging apparatus executes will now be described. However, matters to be clearly understood and expected by one of ordinary skill in the art to which the present invention pertains may be understood by typical implementations even when none of the sources 1210 and 1250, the detectors 1220 and 1260, the processors 1230 and 1270, the displays 1240 and 1280, and the input unit 1290 is specified, and the scope of the present invention is not limited by the titles or physical/logical structures of specified components.

FIG. 13 is a flowchart of a method of operating an X-ray imaging apparatus, according to an embodiment.

An operation of the X-ray imaging apparatus 1200 will now be described, but this description is not be limited to the X-ray imaging apparatus 1200 but may be equally applied to the X-ray imaging apparatus 1205.

In operation S1310 of FIG. 13, the X-ray imaging apparatus 1200 may control a location of at least one of a source that radiates an X ray to an object and a detector that detects an X ray transmitted by the object.

In operation S1320, the X-ray imaging apparatus 1200 may acquire an image captured based on respective locations of the source and the detector.

In operation 51330, the X-ray imaging apparatus 1200 may acquire error information of the captured image due to an error of the location of the at least one of the source and the detector.

In operation S1340, the X-ray imaging apparatus 1200 may display information about correction of the location error of the at least one of the source and the detector, based on the captured image and the error information of the captured image.

There may be provided a computer-readable recording medium having recorded thereon a program for the method of executing operations S1310-S1340 described above with reference to FIG. 13.

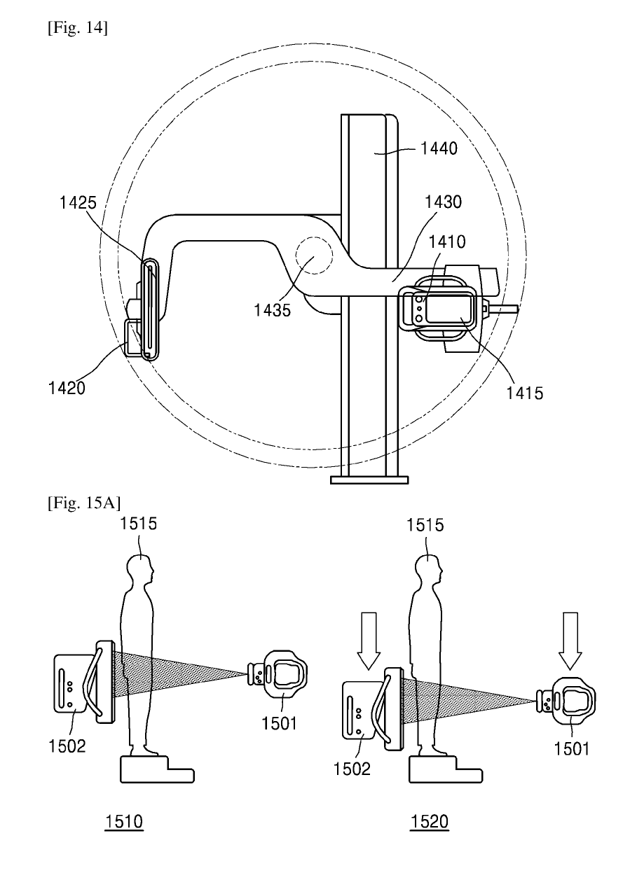

FIG. 14 explains a structure of an X-ray imaging apparatus according to an embodiment.

As shown in FIG. 14, a U-arm type X-ray imaging apparatus may include a source 1410, a detector 1420, an arm 1430, a support 1440, and a processor (not shown). The U-arm type X-ray imaging apparatus may further include an arm connector 1435 connecting the support 1440 to the arm 1430, a source connector 1415 connecting the source 1410 to the arm 1430, and a detector connector 1425 connecting the detector 1420 to the arm 1430. The source connector 1415, the detector connector 1425, and the arm connector 1435 may serve as rotation centers about which the source 1410, the detector 1420, and the arm 1430 rotate, respectively.

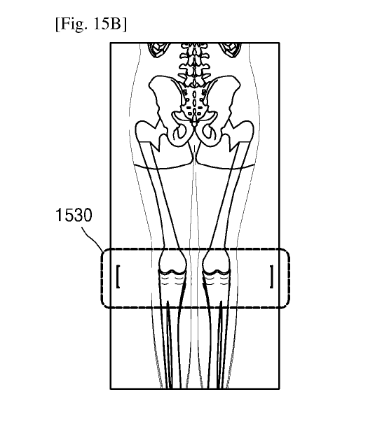

FIG. 15A explains a photographing operation of an X-ray imaging apparatus according to an embodiment.

FIG. 15A illustrates a stepping type photographing operation of the X-ray imaging apparatus 1200. The stepping type photographing operation is to capture an X-ray image while equally moving a source 1501 and a detector 1502.

Referring to FIG. 1510, according to the stepping type photographing method, an X ray is emitted from the source 1501 to the detector 1502 in a direction perpendicular to an X-ray detection surface of the detector 1502. The X-ray imaging apparatus 1200 performs first photography on an object 1515 by detecting an X-ray transmitted by the object 1515.

When the first photography with respect to the object 1515 is concluded, the X-ray imaging apparatus 1200 moves the detector 1502 and the source 1501 and performs second photography as shown in FIG. 1520. During the first photography and the second photography, a radiation angle and distance of the X ray from the source 1501 to the detector 1502 may be constantly maintained, and only heights of the source 1501 and the detector 1502 from the ground may be changed.

FIG. 15B illustrates a synthesis image generated by an X-ray imaging apparatus.

The synthesis image of FIG. 15B is a single image obtained by combining images (an image captured by the first photography and an image captured by the second photography) captured according to the stepping type photographing method described above with reference to FIG. 15A.

In this case, the X-ray imaging apparatus 1200 may acquire an image of a region that is different from a region on an object desired to be acquired, due to a location error of the X-ray imaging apparatus 1200. When the X-ray imaging apparatus 1200 stitches erroneous images, a mismatched region 1530 is generated as shown in FIG. 15B. Accordingly, by correcting the location error of the X-ray imaging apparatus 1200, a match rate and an image quality of the synthesis image obtained by combining the plurality of images may improve.

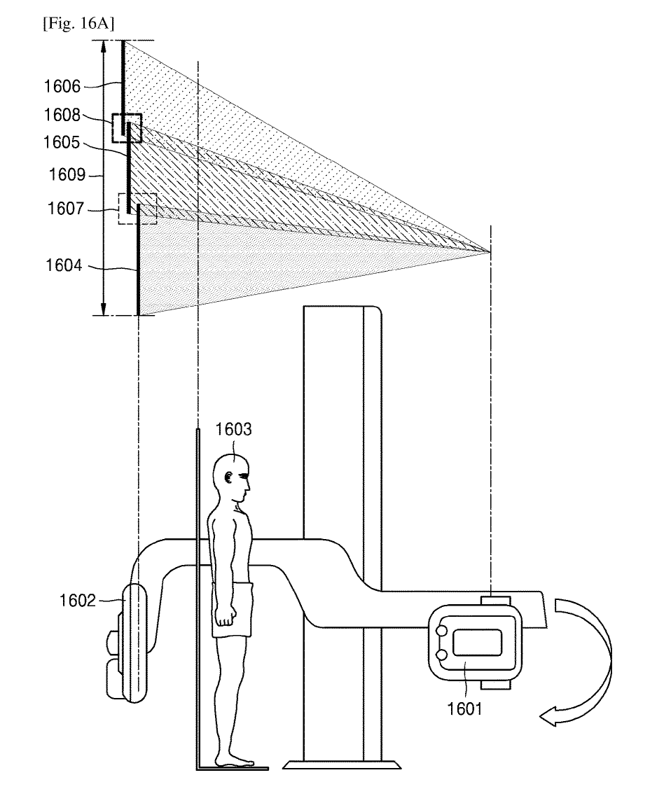

FIG. 16A explains a photographing operation of an X-ray imaging apparatus according to an embodiment.

Referring to FIG. 16A, in the X-ray imaging apparatus 1200, a source emits an X ray to an object, and a detector detects a difference between intensities of X-rays respectively transmitted by the object, thereby acquiring an image.

As shown in FIG. 16A, the X-ray imaging apparatus 1200 may control locations of the source and the detector so that a plurality of images are captured to be overlapped with one another by a predetermined length. The source and the detector may be connected to an arm, and the X-ray imaging apparatus 1200 may control the locations of the source and the detector by controlling the angle of the arm. The term “angle of an arm” denotes an angle formed by the arm with respect to an X-ray detection surface of the detector. The X-ray imaging apparatus 1200 may control the angle of the arm to be equal to an X-ray radiating angle of the source.

For example, the X-ray imaging apparatus 1200 may photograph the object by increasing the angle of the arm 10 degrees at a time with respect to a first location of the detector. As the angle and height of the arm are controlled, the detector is moved farther from the object, and thus the detector is pushed back.

As shown in FIG. 16A, the X-ray imaging apparatus 1200 acquires a first image 1604 at the first location of the detector, acquires a second image 1605 at a second location of the detector, and acquires a third image 1606 at a third location of the detector. The detector being pushed back when the detector is moved from the first location to the second location is indicated by reference numeral 1607, and the detector being pushed back when the detector is moved from the second location to the third location is indicated by reference numeral 1608. As the object and the detector become distant from each other, a region of the object detected by the detector enlarges. As the object and the detector become closer to each other, the region of the object detected by the detector diminishes. The X-ray imaging apparatus 1200 may generate a single synthesis image 1607 by combining the first image 1604, the second image 1605, and the third image 1606 with one another.

FIG. 16B illustrates a synthesis image generated by an X-ray imaging apparatus.

The synthesis image FIG. 16B is a single image obtained by combining images (a first image captured at a first location of a detector and a second image captured at a second location of the detector) captured according to the photographing method described above with reference to FIG. 16A.

In this case, the X-ray imaging apparatus 1200 may acquire an image of a region that is different from a region on an object desired to be acquired, due to a location error of the X-ray imaging apparatus 1200. In detail, although the X-ray imaging apparatus 1200 controls the detector to be positioned at the first location, the X-ray imaging apparatus 1200 actually positions the detector at a location slightly deviating from the first location, thereby generating an error. Thus, due to the location error of the X-ray imaging apparatus 1200, a magnification ratio of a predicted image to be acquired by positioning the detector at the first location is different from a magnification ratio of an actual image. When the X-ray imaging apparatus 1200 stitches erroneous images, a mismatched region 1620 is generated as shown in FIG. 16B. Accordingly, by correcting the location error of the X-ray imaging apparatus 1200, a match rate and an image quality of the synthesis image obtained by combining the plurality of images may be improved.

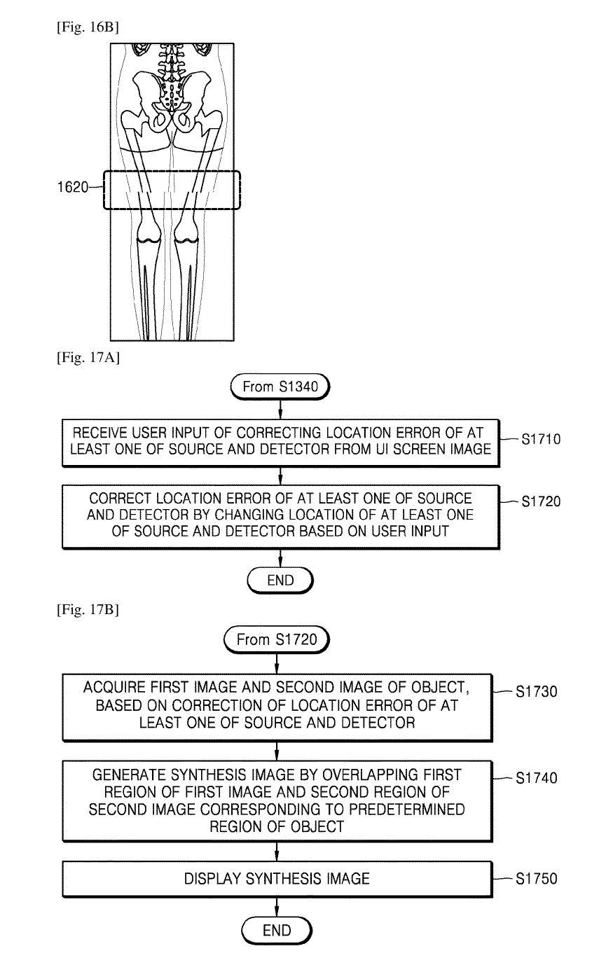

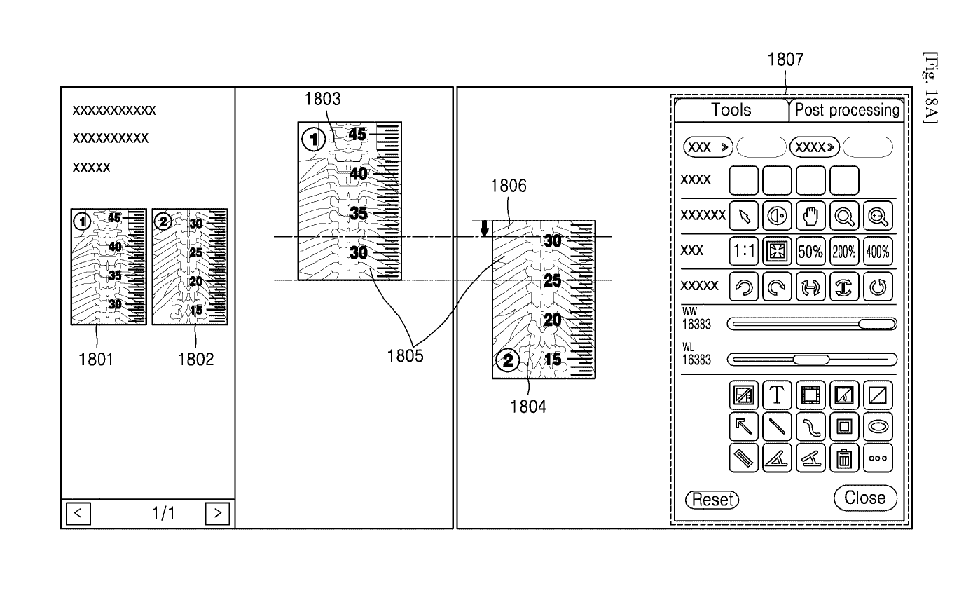

FIG. 17A is a flowchart of a method of operating an X-ray imaging apparatus, according to another embodiment.

In operation S1710 of FIG. 17A, the X-ray imaging apparatus 1205 may receive a user input of correcting at least one of the location errors of the source and the detector from a UI screen image.

In operation S1720, the X-ray imaging apparatus 1205 may correct the at least one of the location errors of the source and the detector, based on the user input. The X-ray imaging apparatus 1205 may correct the at least one location error by changing at least one of the locations of the source and the detector, based on the user input.

FIG. 17B is a flowchart of a method of operating an X-ray imaging apparatus, according to another embodiment.

In operation 51730 of FIG. 17B, the X-ray imaging apparatus 1205 may acquire a first image and a second image of the object, based on correction of the at least one of the locations of the source and the detector. The first image and the second image are images in which the location error correction of the X-ray imaging apparatus 1205 has been reflected.

In operation S1740, the X-ray imaging apparatus 1205 may generate a synthesis image by overlapping a first region of the first image and a second region of the second image corresponding to a predetermined region of the object. In this case, the X-ray imaging apparatus 1205 may generate a synthesis image having no mismatched regions and having an improved quality.

In operation 51750, the X-ray imaging apparatus 1205 may display the synthesis image.

There may be provided a computer-readable recording medium having recorded thereon a program for executing the methods of operating the X-ray imaging apparatus 1205 described above with reference to FIGS. 17A and 17B.

FIG. 18A explains a method of correcting a location error by using a plurality of captured images via a UI screen image, according to an embodiment.

The X-ray imaging apparatus 1205 may correct a location error generated by the X-ray imaging apparatus 1205 by using a first image and a second image acquired by the X-ray imaging apparatus 1205. The X-ray imaging apparatus 1205 may display a UI screen image for correcting the location error.

Referring to FIG. 18A, the X-ray imaging apparatus 1205 may acquire a first image 1801 and a second image 1802 according to the photographing method described above with reference to FIG. 15A. The X-ray imaging apparatus 1205 controls locations of a source and a detector and photographs an object based on the controlled locations.

The X-ray imaging apparatus 1205 photographs the object such that an overlapped region exists between images to achieve continuity of the images. In this case, the X-ray imaging apparatus 1205 may set the length of the overlapped region to be 5 mm, and acquire a first image 1801 and a second image 1802 so that the overlapped region therebetween is 5 mm. When the length of the overlapped region is set to exceed a predetermined value, the number of algorithm calculations during matching between images increases. As the number of overlapped regions increases, the number of joints of a spine bone increases. Thus, the risk of misdiagnosis exists. Thus, the X-ray imaging apparatus 1205 may set a reference value for the overlapped region. When a value of the overlapped region exceeds the reference value, the X-ray imaging apparatus 1205 may correct the overlapped region automatically or manually (for example, a user input).

Referring to FIG. 18A, due to a location error of the X-ray imaging apparatus 1205, the length of the overlapped region between the first image 1801 and the second image 1802 may exceed 5 mm. The X-ray imaging apparatus 1205 may receive a user input of correcting the location error, via a UI screen image.

The X-ray imaging apparatus 1205 may receive an input of correcting a section of the overlapped region between the first image 1801 and the second image 1802. The UI screen image may include an icon 1807 for manipulating the first image 1801 and the second image 1802.

The X-ray imaging apparatus 1205 may receive a manipulation signal generated due to various input tools or a user touch input. The X-ray imaging apparatus 1205 may receive an input of moving the second image 1802 by a hand or a physical tool of a user. Referring to FIG. 18A, a UI may receive an input 1806 of moving the second image 1802 downwards by 5 mm. The X-ray imaging apparatus 1205 may move the second image 1802 downwards by 5 mm so that respective overlapping regions of the first image 1801 and the second image 1802 generated due to the location error of the X-ray imaging apparatus 1205 may match with each other.

By performing correction via the UI screen image so that the overlapping regions of the first image 1801 and the second image 1802 match with each other, the X-ray imaging apparatus 1205 may correct the location error of the X-ray imaging apparatus 1205 and prevent a location error from being generated by the next photography.

FIG. 18B explains a method of correcting a location error by using a plurality of captured images via a UI screen image, according to another embodiment.

Referring to FIG. 18B, the X-ray imaging apparatus 1205 may acquire a first image 1811 and a second image 1812 according to the photographing method described above with reference to FIG. 15A.

Referring to FIG. 18B, due to a location error of the X-ray imaging apparatus 1205, the length of an overlapped region between the first image 1811 and the second image 1812 may be less than a reference length by 4 mm. The reference length is a length of the overlapped region that is minimally necessary for matching the first image 1811 with the second image 1810. As the length of the overlapped region is less than a predetermined value or the overlapped region becomes smaller, the same part of an object is not included in the overlapped region, and thus a case where matching is impossible may occur. Thus, the X-ray imaging apparatus 1205 may set a reference value for the overlapped region. When a value of the overlapped region is less than the reference value, the X-ray imaging apparatus 1205 may correct the overlapped region automatically or manually (for example, a user input).

The X-ray imaging apparatus 1205 may receive an input of correcting a section of the overlapped region between the first image 1811 and the second image 1812. The X-ray imaging apparatus 1205 may receive an input 1816 of moving the second image 1812 upwards by 4 mm. The X-ray imaging apparatus 1205 may move the second image 1812 upwards by 4 mm so that respective overlapping regions of the first image 1811 and the second image 1812 generated due to the location error of the X-ray imaging apparatus 1205 may match with each other.

FIG. 19 explains a synthesis image in which location error correction of an X-ray imaging apparatus has been reflected, and a synthesis image in which location error correction of an X-ray imaging apparatus has not yet been reflected, according to an embodiment.

The X-ray imaging apparatus 1205 may acquire an X-ray image of an object by combining a plurality of X-ray images with one another. The X-ray imaging apparatus 1205 may acquire a first image captured based on a first location of a source and a first location of a detector, and a second image captured based on a second location of the source and a second location of the detector.

Due to a location error of the X-ray imaging apparatus 1205, the first image or the second image may have an error. The location error of the X-ray imaging apparatus 1205 may be generated due to errors between respective actual locations of the source and the detector and respective predicted locations of the source and the detector. The location error of the X-ray imaging apparatus 1205 may also be generated due to an error of an Image to Object Distance (IOD) or may be generated from the fact that the location of the object is not fixed.

Since the first image or the second image has an error, even when the first image and the second image are synthesized by overlapping respective overlapping regions of the first image and the second image, an overlapped region 1911 between the first image and the second image has a mismatched region. The X-ray imaging apparatus 1205 may display, on a screen image 1910, a synthesis image including a mismatched region and information representing that matching between the first image and the second image has failed.

As described above with reference to FIGS. 18A and 18B, the X-ray imaging apparatus 1205 may correct the location error of the X-ray imaging apparatus 1205 by using the first image and the second image. Due to the location error correction, the X-ray imaging apparatus 1205 may acquire a third image and a fourth image each having no location errors at the next photography. The X-ray imaging apparatus 1205 may acquire a matched synthesis image by overlapping respective overlapping regions of the third image and the fourth image. In other words, the X-ray imaging apparatus 1205 may acquire a synthesis image in which respective overlapping regions 1921 of the third image and the fourth image are matched with each other. The X-ray imaging apparatus 1205 may display, on a screen image 1920, the matched synthesis image and information representing that matching between the third image and the fourth image has succeeded.

FIG. 20A explains a method of correcting a location error by using a plurality of captured images via a UI screen image, according to another embodiment.

According to the photographing method described above with reference to FIG. 16A, the X-ray imaging apparatus 1205 may control respective locations of a source and a detector and photograph an object based on the controlled locations to thereby acquire a first image 2001 and a second image 2002.

As described above with reference to FIG. 16A, the detector is pushed back according to an angle of an arm. Thus, images captured according to angles of the arm have different magnification ratios.

Theoretically, it is assumed that a first image captured according to a first angle of the arm has a first magnification ratio and a second image captured according to a second angle of the arm has a second magnification ratio. A magnification ratio may be calculated from a ratio of an SID to an SOD. However, due to a location error of the X-ray imaging apparatus 1205, the first image may have another magnification ratio instead of the first magnification ratio, and the second image may have another magnification ratio instead of the second magnification ratio. The location error of the X-ray imaging apparatus 1205 may be generated due to errors between respective actual locations of the source and the detector and respective predicted locations of the source and the detector. The location error of the X-ray imaging apparatus 1205 may also be generated due to an error of an IOD. The location error of the X-ray imaging apparatus 1205 may also be generated due to the environments of an installation place of the X-ray imaging apparatus 1205 or a location error of a jig stopper that moves the object.

The X-ray imaging apparatus 1205 may receive a user input of correcting the location error of the X-ray imaging apparatus 1205, via a UI screen image. The X-ray imaging apparatus 1205 may receive a user input of correcting the magnification ratio of the first image to a magnification ratio of a first predicted image for the first image and the magnification ratio of the second image to a magnification ratio of a second predicted image for the second image, and match the first image with the second image according to the user input. According to the corrections of the magnifications of the first and second images, the X-ray imaging apparatus 1205 may correct the location error of the X-ray imaging apparatus 1205.

When the X-ray imaging apparatus 1205 matches the first image with the second image in response to a user input of making the magnification ratio of the first image identical to the magnification ratio of the second image, the X-ray imaging apparatus 1205 may correct the location error thereof.

The X-ray imaging apparatus 1205 may receive a user input of correcting the location error, via a UI screen image. The X-ray imaging apparatus 1205 may receive an input of correcting the magnification ratios of the first image and the second image. The UI screen image may include an icon for manipulating the first image and the second image.

The X-ray imaging apparatus 1205 may receive a manipulation signal generated due to various input tools or a user touch input. The X-ray imaging apparatus 1205 may receive an input of increasing or decreasing the magnification ratio of the first image or the magnification ratio of the second image by a hand or a physical tool of a user.

By performing correction such that the magnification ratios of the first image and the second image are identical to each other, the X-ray imaging apparatus 1205 may correct the location error of the X-ray imaging apparatus 1205 and prevent a location error from being generated by the next photography.

FIG. 20B explains a synthesis image in which location error correction of an X-ray imaging apparatus has been reflected, and a synthesis image in which location error correction of an X-ray imaging apparatus has not yet been reflected, according to an embodiment.

According to the photographing method described above with reference to FIG. 16A, the X-ray imaging apparatus 1205 may control respective locations of a source and a detector and photograph an object based on the controlled locations to thereby acquire a first image and a second image.

Due to a location error of the X-ray imaging apparatus 1205, the first image or the second image may have a magnification error. Thus, when the X-ray imaging apparatus 1205 makes the magnification ratio of the first image identical to the magnification ratio of the second image and generates a synthesis image by overlapping a first region of the first image with a second region of the second image, a mismatched region may be generated in an overlapped region 2011 between the first image and the second image due to the location error of the X-ray imaging apparatus 1205.

As described above with reference to FIG. 20A, the X-ray imaging apparatus 1205 may correct the location error of the X-ray imaging apparatus 1205 by using the first image and the second image. Due to the location error correction, the X-ray imaging apparatus 1205 may acquire a third image and a fourth image each having no location errors at the next photography. The X-ray imaging apparatus 1205 may acquire a matched synthesis image by overlapping respective overlapping regions of the third image and the fourth image. In other words, the X-ray imaging apparatus 1205 may acquire a synthesis image in which respective overlapping regions 2021 of the third image and the fourth image are matched with each other.

FIG. 21 is a flowchart of a method of operating an X-ray imaging apparatus, according to an embodiment. In detail, FIG. 21 is a flowchart of a method in which an X-ray imaging apparatus acquires error information of a captured image.

In operation 52110, the X-ray imaging apparatus 1205 may detect a region of a collimator corresponding to an X-ray irradiated region of the collimator from the captured image.

In operation 52120, the X-ray imaging apparatus 1205 may acquire information about the area of the collimator region and the central point of the collimator region, from the captured image.

In operation 52130, the X-ray imaging apparatus 1205 may compare the information about the area of the collimator region and the central point of the collimator region with preset information about the area of the collimator region and the central point of a detector to thereby acquire the error information of the captured image. The central point of a detector may be a center value of a detector region in a coordinate system representing a location of the detector.

When an image is captured when the central point of the collimator region is not identical to the central point of the detector, a region of interest of an object may deviate from a photographing range. Thus, the X-ray imaging apparatus 1205 may receive a user input of correcting the central point of the collimator region and the central point of the detector to be identical to each other. The X-ray imaging apparatus 1205 may compare the area of the collimator region acquired from the captured image with the area of a preset collimator region and the central point of the collimator region acquired from the captured image with the central point of the detector and thus may acquire error information as a result of the comparison.

FIGS. 22A and 22B explain a method of correcting a location error by using a captured image via a UI screen image, according to an embodiment.

Referring to FIG. 22A, the X-ray imaging apparatus 1205 may compare a central point 2202 of a collimator with a central point 2203 of a detector and may display a result of the comparison on a screen image 2205. The central point 2202 of the collimator is a central point of a region 2201 on an image that corresponds to an X-ray irradiated region of the collimator. In other words, the central point 2202 of the collimator may be a central point of a collimation blade. During capturing of an X-ray image, the X-ray image may be accurately captured only when the central point 2202 of the collimator is identical with the central point 2203 of the detector.

The X-ray imaging apparatus 1205 may receive at least one of a user input of adjusting the area of a collimator region and a user input of adjusting the central point of the collimator region, and may adjust at least one of the area of the collimator region and the central point of the collimator region according to the received user input.

The X-ray imaging apparatus 1205 may receive a user input of making the central point of the collimator region and the central point of the detector be identical to each other via a UI screen image.

In detail, the UI screen image may include an icon 1807 for setting a central point of the collimator region or moving the central point. The X-ray imaging apparatus 1205 may receive an input of correcting the central point of the collimator region to the central point of a preset collimator region by using a hand or a physical tool of a user. In detail, the X-ray imaging apparatus 1205 may receive an input of moving the central point 2202 of the collimator region to the central point of the preset collimator region. A user move the central point 2202 of the collimator region to the central point of the preset collimator region by using a drag-and-drop function of a stylus pen on the screen image 2205. It will be understood by one of ordinary skill in the art to which this embodiment pertains that the central point 2202 of the collimator region may be moved to the central point of the preset collimator region according to other methods than the aforementioned method.

When a difference between the central point 2202 of the collimator and the central point 2203 of the detector is very small, the X-ray imaging apparatus 1205 may be leveled with the ground by moving the central point of the collimator. On the other hand, when a difference between the central point 2202 of the collimator and the central point 2203 of the detector is large, the X-ray imaging apparatus 1205 may be re-mounted to be leveled with the ground.

Referring to FIG. 22B, the X-ray imaging apparatus 1205 may display a screen image 2212 on which a collimator region 2201 on an image corresponding to a collimator and a preset collimator region 2211 are displayed. During capturing of an X-ray image, the X-ray image may be accurately captured only when the area of the collimator region 2201 is identical with the area of the preset collimator region 2211.

The X-ray imaging apparatus 1205 may make the area of the collimator region 2201 be identical with the area of the preset collimator region 2211 by making the central point of the collimator be identical with the central point of the detector and making the area of the collimator region 2201 be identical with the area of the preset collimator region 2211.

The X-ray imaging apparatus 1205 may receive a user input of making the collimator region 2201 and the preset collimator region 2211 be identical to each other via a UI screen image. For example, a user may make the collimator region 2201 be identical with the preset collimator region 2211 by using a drag-and-drop function of a stylus pen on the screen image 2212. It will be understood by one of ordinary skill in the art to which this embodiment pertains that the collimator region 2201 may be made identical with the preset collimator region 2211 according to other methods than the aforementioned method.

By making the collimator region 2201 and the preset collimator region 2211 be identical with each other, image precision of the X-ray imaging apparatus may improve.

FIG. 23 is a flowchart of a method of operating an X-ray imaging apparatus, according to an embodiment. In detail, FIG. 23 is a flowchart of a method in which an X-ray imaging apparatus acquires error information of a captured image.

In operation 52310, the X-ray imaging apparatus 1205 may detect a region of a collimator corresponding to an X-ray irradiated region of the collimator from the captured image.

In operation 52320, the X-ray imaging apparatus 1205 may acquire first captured coordinate values representing coordinate values of a plurality of vertices of the collimator region from the captured image, and may also acquire first predicted coordinate values representing predicted coordinate values of the plurality of vertices of the collimator region. The first predicted coordinate values corresponding to the first captured coordinate values may be calculated from a preset collimator region on the collimator.

In operation 52330, the X-ray imaging apparatus 1205 may acquire error information of the captured image by comparing the first predicted coordinate values with the first captured coordinate values.

FIG. 24 explains a method of correcting a location error by using a captured image via a UI screen image, according to an embodiment.

Referring to FIG. 24, the X-ray imaging apparatus 1205 may detect a collimator region corresponding an X-ray irradiated region of a collimator, and acquire first captured coordinate values 2401, 2402, 2403, and 2404 representing coordinates of a plurality of vertices of the collimator region from the captured image. The X-ray imaging apparatus 1205 may acquire first predicted coordinate values representing predicted coordinate values of the plurality of vertices of the collimator region. In detail, the X-ray imaging apparatus 1205 may calculate the first predicted coordinate values corresponding to the first captured coordinate values from a preset collimator region on the collimator. The X-ray imaging apparatus 1205 may display the first captured coordinate values 2401, 2402, 2403, and 2404 on a screen image, and may also display first predicted coordinate values 2411, 2412, 2413, and 2414 of a plurality of vertices of the preset collimator region on the image screen.

The X-ray imaging apparatus 1205 may correct the location error of the X-ray imaging apparatus 1205 by moving the region of the collimator so that the first captured coordinate values 2401, 2402, 2403, and 2404 are identical with the first predicted coordinate values 2411, 2412, 2413, and 2414.

The X-ray imaging apparatus 1205 may receive a user input of moving the region of the collimator so that the first captured coordinate values 2401, 2402, 2403, and 2404 are identical with the first predicted coordinate values 2411, 2412, 2413, and 2414.

The above-described apparatus may be implemented by using a hardware component, a software component, and/or a combination of a hardware component and a software component. For example, the apparatus and the component described in the exemplary embodiments may be implemented by using one or more general-purpose computers or a special-purpose computers such as, for example, a processor, a controller, an arithmetic logic unit (ALU), a digital signal processor, a microcomputer, a field programmable array (FPA), a programmable logic unit (PLU), a microprocessor, or any device that may execute an instruction and respond thereto.

A processor may execute an operating system (OS) and one or more software applications executed on the OS. Also, the processor may access, store, manipulate, process, and generate data in response to execution of software.

For convenience of understanding, though description has been made to the case where one processor is used, a person of ordinary skill in the art will understand that the processor may include a plurality of processing elements and/or processing elements having a plurality of types. For example, the processor may include a plurality of processors, or one processor and one controller. Also, the processor may include a different processing configuration such as a parallel processor.

Software may include a computer program, a code, an instruction, or a combination of one or more of these, and configure the processor to operate as desired, or instruct the processor independently or collectively.

Software and/or data may be embodied permanently or temporarily in a certain type of a machine, a component, a physical device, virtual equipment, a computer storage medium or device, or a transmitted signal wave in order to allow the processor to analyze the software and/or data, or to provide an instruction or data to the processor. Software may be distributed on a computer system connected via a network, and stored and executed in a distributed fashion. Software and data may be stored in one or more non-transitory computer-readable recording media.

The methods according to exemplary embodiments may be embodied in the form of program commands executable through various computer means, which may be recorded on a non-transitory computer-readable recording medium. The non-transitory computer-readable recording medium may include program commands, data files, and data structures either alone or in combination. The program commands recorded on the non-transitory computer-readable recording medium may be those that are especially designed and configured for the inventive concept, or may be those that are known and available to computer programmers skilled in the art.

Examples of the non-transitory computer-readable recording medium include magnetic recording media such as hard disks, floppy disks, and magnetic tapes, optical recording media such as CD-ROMs and DVDs, magneto-optical recording media such as floptical disks, and hardware devices such as ROMs, RAMs, and flash memories that are especially configured to store and execute program commands.

Examples of the program commands include machine language codes that may be generated by a compiler, and high-level language codes that may be executed by a computer by using an interpreter.

The above hardware device may be configured to operate as one or more software modules in order to perform an operation of an exemplary embodiment, and vice versa.

Though the exemplary embodiments have been described by a limited number of exemplary embodiments and drawings, a person of ordinary skill in the art will make various modifications and changes from the above exemplary embodiments. For example, even when the described technologies are performed in an order different from the described method and/or components such as the described system, structure, apparatus, and circuit are coupled or combined in a form different from the described method, or replaced by other components or equivalents thereof, a proper result may be accomplished.

Therefore, the scope of the inventive concept should not be limited and determined by the described exemplary embodiments, but should be determined by not only the following claims but also equivalents thereof.

1. A medical image processing apparatus comprising:
a processor configured to:
acquire a first image and a second image captured by radiating object with an X-ray;
generate a synthesis image by overlapping a first region of the first image with a second region of the second image; and
determine matching accuracy representing a degree to which the first region and the second region match with each other; and
a display configured to display the matching accuracy and the synthesis image.

2. The medical image processing apparatus of claim 1, wherein the display displays at least one of information about a length of an overlapped region between the first image and the second image on the synthesis image or information about a location of the overlapped region on the synthesis image.

3. The medical image processing apparatus of claim 1, wherein, when the first region of the first image and the second region of the second image do not match with each other, the display is configured to distinguishably display a predetermined portion corresponding to a mismatched region between the first region of the first image and the second region of the second image.

4. The medical image processing apparatus of claim 1, wherein:
the display is configured to display, together with the synthesis image, a marker indicating a location of an overlapped region between the first image and the second image, and
the processor is configured distinguishably set at least one of a color, shape, or pattern of the marker, based on the matching accuracy.

5. The medical image processing apparatus of claim 1, further comprising:
an input unit configured to receive a user input for correcting a range of at least one of the first region of the first image and the second region of the second image,
wherein the processor is configured to correct the range of the at least one of the first region and the second region, based on the user input, and re-generates a synthesis image by using a result of the correction.

6. A medical image processing method comprising:
acquiring a first image and a second image captured by radiating an object with an X-ray;
generating a synthesis image by overlapping a first region of the first image with a second region of the second image;
determining matching accuracy representing a degree to which the first region and the second region match with each other; and
displaying the matching accuracy and the synthesis image.

7. An X-ray imaging apparatus comprising:
a source configured to radiate an object with an X-ray;
a detector configured to detect an X-ray from the object;
a processor configured to:
control a location of at least one of the source and the detector;
acquire an image captured based on the location of the source and the location of the detector; and
acquire error information of the captured image due to a location error of at least one of the source or the detector from the captured image; and
a display configured to display information about a correction of the at least one location error based on the captured image and the error information of the captured image.

8. The X-ray imaging apparatus of claim 7, wherein the processor is configured to:
generate a predicted image based on the location of the source and the location of the detector;
compare the captured image with the predicted image; and
acquire the error information of the captured image according to a result of the comparison.

9. The X-ray imaging apparatus of claim 8, further comprising an input unit configured to receive a user input of correcting the at least one location error,
wherein the processor is configured to correct the at least one location error by changing a location of at least one of the source or the detector based on the user input.

10. The X-ray imaging apparatus of claim 9, wherein:
the processor is configured to:
acquire a first image and a second image of the object captured based on the correction of the at least one location error; and
overlap a first region of the first image with a second region of the second image,
the first and second regions correspond to a predetermined region of the object, to generate a synthesis image, and
the display is configured to display the synthesis image.

11. The X-ray imaging apparatus of claim 9, wherein:
the processor is configured to:
detect an error due to a difference between magnification ratios of the captured image and the predicted image; and
correct the magnification ratio of the captured image to the magnification ratio of the predicted image based on the user input; and
the input unit is configured to receive a user input for correcting the error due to the difference between the magnification ratios.

12. The X-ray imaging apparatus of claim 9, wherein the processor is configured to:
detect a collimator region corresponding to an X-ray irradiated region of a collimator from the captured image;
acquire information about an area of the collimator region and a central point of the collimator region from the captured image; and
compare the information about the area of the collimator region and the central point of the collimator region with preset information about the area of the collimator region and a central point of the detector to acquire the error information of the captured image.

13. The X-ray imaging apparatus of claim 12, wherein:
the input unit is configured to receive at least one of a user input of adjusting the area of the collimator region and a user input of adjusting the central point of the collimator region, and
the processor is configured to adjust at least one of the area of the collimator region and the central point of the collimator region according to the received user input.

14. The X-ray imaging apparatus of claim 9, wherein:
the processor is configured to:
detect a collimator region corresponding to an X-ray irradiated region of a collimator from the captured image;
acquire, from the captured image, a first captured coordinate values representing coordinate values of a plurality of vertices of the collimator region;
calculate a first predicted coordinate values representing predicted coordinate values of the plurality of vertices of the collimator region based on the location of the source and the location of the detector; and
acquire error information of the captured image with respect to the collimator region by comparing the first predicted coordinate values with the first captured coordinate values; and
the display is configured to display the error information with respect to the collimator region.

15. The X-ray imaging apparatus of claim 14, wherein:
the input unit is configured to receive a user input of moving the collimator region so that the first captured coordinate values are identical with the first predicted coordinate values based on the error information; and
the processor is configured to control the source to move the collimator region based on the user input.
